(19) United States
(12) Patent Application Publication
(10) Pub. No.: US 2026/0285931 A1
(43) Pub. Date: Sep. 24, 2026

(54) ENGINEERED IL-7 VARIANTS AND METHODS OF USE THEREOF

(71) Applicant: FBD BIOLOGICS LIMITED, Hong Kong (CN)

(72) Inventors: Huan-Ching LIN, Taipei (TW); Yi Hsuan LU, Taipei (TW); Yi-Hsuan LI, Taipei (TW); Chi-Ling TSENG, Taipei (TW); Zong Sean JUO, Taipei (TW)

(21) Appl. No.: 19/473,707

(22) PCT Filed: Jun. 6, 2024
(86) PCT No.: PCT/CN2024/097763

§ 371 Date: Oct. 8, 2025

Related U.S. Application Data
(60) Provisional application No. 63/471,694, filed on Jun. 7, 2023.

(51) Int. Cl. C07K 14/54; A61K 38/00; A61P 35/00

(52) U.S. Cl. CPC C07K 14/5418; A61P 35/00; A61K 38/00

(57) ABSTRACT

The engineered IL-7 variants, and methods of use thereof are disclosed.

### CROSS-REFERENCE TO RELATED APPLICATION

This disclosure claims priority to and benefit of U.S. Provisional Patent Application Ser. No. 63/471,694, filed Jun. 7, 2023, which is incorporated herein by reference in its entirety.

### SEQUENCE LISTING

This application contains a Sequence Listing that has been submitted electronically as an XML file named “52246-0009WO1_SL_ST26.XML.” The XML file, created on Jun. 5, 2024, is 49,693 bytes in size. The material in the XML file is hereby incorporated by reference in its entirety.

### TECHNICAL FIELD

This disclosure relates to engineered IL-7 variants, and methods of use thereof.

### BACKGROUND

T-cell development occurs through several stages in the thymus. Immature T-cells need to reach sufficient levels because their development has several stages known as positive and negative selection, contributing to the loss of 98% of T-cells. Where regulation of human T cell maturation was achieved by decreasing IL-7 and increasing anti-CD3 levels. These effects are supported by studies demonstrating how intrathymic T cell development is dependent in some part on the presence of IL-7. Specifically, IL-7 along with thymic stromal lymphopoietin seem to be necessary for Treg maturation.

However, wildtype IL-7 is relatively unstable. Thus, there is a need to develop cancer therapies targeting the IL-7 pathway with improved stability.

### SUMMARY

This disclosure relates to engineered IL-7 variants, and methods of use thereof. In some embodiments, the engineered IL-7 variants have a rearranged helices A-D, such that helix D is at the N-terminus. In some embodiments, the IL-7 variants described herein can maintain the orientation of helices A-D as compared to wildtype IL-7. In some embodiments, the IL-7 variants have a comparable binding affinity to CD127 and functional potency (e.g., inducing STAT5 phosphorylation on primary T cells and/or inducing primary T cell proliferation) as compared to wildtype IL-7. The engineered IL-7 variants described herein can have “tunable” potencies and affinities, which can facilitate development of cancer therapies, lymphopenia therapies, and vaccines.

In some embodiments, the engineered IL-7 polypeptide can bind to CD127 (e.g., human CD127) with an EC50 that is less than 10%, less than 20%, less than 30%, less than 40%, less than 50%, less than 60%, less than 70%, less than 80%, less than 90%, less than 1-fold, less than 2-fold, less than 3-fold, less than 4-fold, or less than 5-fold as compared to a reference polypeptide comprising wildtype IL-7. In some embodiments, the engineered IL-7 polypeptide can bind to CD127 (e.g., human CD127), e.g., with a KD of less than 1×10⁻⁷ M, less than 1×10⁻⁸ M, or less than 1×10⁻⁹ M.

In some embodiments, the engineered IL-7 polypeptide can induce primary T cell proliferation, e.g., with an EC50 that is less than 50%, less than 60%, less than 70%, less than 80%, less than 90%, less than 1-fold, less than 2-fold, less than 3-fold, less than 4-fold, less than 5-fold, less than 10-fold, less than 20-fold, less than 30-fold, less than 40-fold, less than 50-fold, or less than 100-fold as compared to that of a reference polypeptide comprising wildtype IL-7.

In one aspect, the disclosure is related to an engineered IL-7 polypeptide comprising: a helix D, a helix A, a helix B, and a helix C, in some embodiments, the helix D and helix A are linked via a DA loop sequence. In some embodiments, the engineered IL-7 polypeptide maintains the orientation of helix A, helix B, helix C, and helix D of a wildtype IL-7 polypeptide. In some embodiments, the engineered IL-7 polypeptide can bind to CD127 (e.g., human CD127) and/or CD132 (e.g., human CD132). In some embodiments, the engineered IL-7 polypeptide can induce STAT5 phosphorylation on primary T cells and/or induce primary T cell proliferation. In some embodiments, the engineered IL-7 polypeptide comprises from N-terminus to C-terminus: (a) a helix D fragment amino acid sequence; and (b) a helix ABC fragment amino acid sequence, in some embodiments, the helix D fragment and the helix ABC fragment are linked by the DA loop sequence. In some embodiments, the helix D fragment amino acid sequence is at least 80%, 85%, 90%, or 95% identical to SEQ ID NO: 50 and/or the helix ABC fragment amino acid sequence is at least 80%, 85%, 90%, or 95% identical to SEQ ID NO: 51 or SEQ ID NO: 52.

In one aspect, the disclosure is related to an engineered IL-7 polypeptide comprising from N-terminus to C-terminus: (a) a helix D sequence that is at least 80%, 85%, 90%, or 95% identical to SEQ ID NO: 30; (b) a helix A sequence that is at least 80%, 85%, 90%, or 95% identical to SEQ ID NO: 31; (c) a helix B sequence that is at least 80%, 85%, 90%, or 95% identical to SEQ ID NO: 32; and (d) a helix C sequence that is at least 80%, 85%, 90%, or 95% identical to SEQ ID NO: 33. In some embodiments, the engineered IL-7 polypeptide described herein comprises a DA loop sequence between the helix D sequence and the helix A sequence, in some embodiments, the DA loop sequence is at least 80%, 85%, 90%, or 95% identical to any one of SEQ ID NOs: 34-49. In some embodiments, the engineered IL-7 polypeptide comprises an amino acid sequence that is at least 80%, 85%, 90%, or 95% identical to SEQ ID NO: 50. In some embodiments, the engineered IL-7 polypeptide comprises an amino acid sequence that is at least 80%, 85%, 90%, or 95% identical to SEQ ID NO: 51. In some embodiments, the engineered IL-7 polypeptide comprises an amino acid sequence that is at least 80%, 85%, 90%, or 95% identical to any one of SEQ ID NOs: 3-18. In some embodiments, the engineered IL-7 polypeptide comprises an amino acid sequence that is at least 80%, 85%, 90%, or 95% identical to SEQ ID NO: 52. In some embodiments, the engineered IL-7 polypeptide comprises an amino acid sequence that is at least 80%, 85%, 90%, or 95% identical to any one of SEQ ID NOs: 21-27.

In one aspect, the disclosure is related to an engineered IL-7 polypeptide, in some embodiments, the engineered IL-7 polypeptide comprises mutations at one or more glycosylation sites. In some embodiments, the engineered IL-7 polypeptide described herein comprises one or more of the following: (a) the amino acid that corresponds to N95 of SEQ ID NO: 1 is Q, E, or A; (b) the amino acid that corresponds to N116 of SEQ ID NO: 1 is Q, E, or A; and/or (c) the amino acid that corresponds to N141 of SEQ ID NO: 1 is Q, E, or A. In some embodiments, the engineered IL-7 polypeptide described herein comprises one or more of the following: (a) the amino acid that corresponds to N95 of SEQ ID NO: 1 is Q; (b) the amino acid that corresponds to N116 of SEQ ID NO: 1 is Q; and/or (c) the amino acid that corresponds to N141 of SEQ ID NO: 1 is Q. In some embodiments, the engineered IL-7 polypeptide comprises an amino acid sequence that is at least 80%, 85%, 90%, or 95% identical to SEQ ID NO: 19 or SEQ ID NO: 51.

In one aspect, the disclosure is related to a protein construct comprising the engineered IL7 polypeptide described herein. In some embodiments, the protein construct described herein comprises two or more engineered IL-7 polypeptides. In some embodiments, at least two engineered IL-7 polypeptides are identical. In some embodiments, at least two engineered IL-7 polypeptides are different. In some embodiments, the protein construct described herein further comprises an Fc region. In some embodiments, the Fc region is an IgG4 Fc region (e.g., a human IgG4 Fc region with a S228P mutation according to EU numbering). In some embodiments, the Fc region comprises an amino acid sequence that is at least 80%, 85%, 90%, or 95% identical to SEQ ID NO: 28. In some embodiments, the Fc region is an IgG1 Fc region (e.g., with LALA mutations or LALA-PG mutations). In some embodiments, the engineered IL-7 polypeptide is connected to the Fc region via a linker peptide. In some embodiments, the linker peptide comprises an amino acid sequence that is at least 80%, 85%, 90%, or 95% identical to SEQ ID NO: 29.

In one aspect, the disclosure is related to a protein construct comprising a first fusion polypeptide comprising the engineered IL-7 polypeptide described herein, a first CH2 domain, and a first CH3 domain; and a second fusion polypeptide comprising a second CH2 domain, and a second CH3 domain; in some embodiments, the first fusion polypeptide and the second fusion polypeptide associate with each other, forming a dimer. In some embodiments, the second fusion polypeptide further comprises a second engineered IL-7 polypeptide.

In one aspect, the disclosure is related to a pharmaceutical composition comprising the engineered IL-7 polypeptide or the protein construct described herein; and a pharmaceutically acceptable carrier.

In one aspect, the disclosure is related to a nucleic acid encoding the engineered IL-7 polypeptide or the protein construct described herein. In one aspect, the disclosure is related to a vector comprising the nucleic acid described herein. In one aspect, the disclosure is related to a cell comprising the nucleic acid described herein. In some embodiments, the cell is a CHO cell.

In one aspect, the disclosure is related to a method of producing an engineered IL-7 polypeptide or a protein construct comprising the engineered IL-7 polypeptide, the method comprising (a) culturing the cell described herein under conditions sufficient for the cell to produce the engineered IL-7 polypeptide or the protein construct; and (b) collecting the engineered IL-7 polypeptide or the protein construct produced by the cell.

In one aspect, the disclosure is related to a method of treating a subject having cancer, the method comprising administering a therapeutically effective amount of a composition comprising the engineered IL-7 polypeptide or the protein construct described herein, to the subject. In some embodiments, the subject has a solid tumor or a hematologic cancer. In some embodiments, the cancer is lung cancer, melanoma, colorectal cancer, glioma, pancreatic cancer, lymphoma, leukemia, prostate cancer, renal cell carcinoma (RCC), hepatocellular carcinoma, cholangiocarcinoma, gallbladder cancer, gastric cancer, endometrial carcinoma, ovarian cancer, bladder cancer, or glioblastoma.

In one aspect, the disclosure is related to a method of decreasing the rate of tumor growth, the method comprising contacting a tumor cell with an effective amount of a composition comprising the engineered IL-7 polypeptide or the protein construct described herein. In one aspect, the disclosure is related to a method of killing a tumor cell, the method comprising contacting a tumor cell with an effective amount of a composition comprising the engineered IL-7 polypeptide or the protein construct described herein.

As used herein, the term “engineered IL-7 polypeptide” refers to a polypeptide derived from a wildtype IL-7 polypeptide or a portion thereof (e.g., without the signal peptide) with one or more mutations (e.g., insertions, deletions, or substitutions). In some embodiments, the engineered IL-7 polypeptide comprises or consists of the four α-helical domains (helices) of IL-7. In some embodiments, the engineered IL-7 polypeptide comprises or consists of helix A, helix B, helix C, helix D, or a fragment thereof of wildtype IL-7. In some embodiments, the engineered IL-7 polypeptide maintains the orientation of helix A, helix B, helix C, and helix D, e.g., by substantially preserving the interactions with CD127 and/or CD132.

As used herein, the term “protein construct” refers to a complex having one or more polypeptides. In some embodiments, the protein construct has two or more polypeptides, wherein the polypeptides can associate with each other, forming a dimer or a multimer.

As used herein, the term “cancer” refers to cells having the capacity for uncontrolled autonomous growth. Examples of such cells include cells having an abnormal state or condition characterized by rapidly proliferating cell growth. The term is meant to include cancerous growths, e.g., tumors; oncogenic processes, metastatic tissues, and malignantly transformed cells, tissues, or organs, irrespective of histopathologic type or stage of invasiveness. Also included are malignancies of the various organ systems, such as respiratory, cardiovascular, renal, reproductive, hematological, neurological, hepatic, gastrointestinal, and endocrine systems; as well as adenocarcinomas which include malignancies such as most colon cancers, renal-cell carcinoma, prostate cancer and/or testicular tumors, non-small cell carcinoma of the lung, and cancer of the small intestine. Cancer that is “naturally arising” includes any cancer that is not experimentally induced by implantation of cancer cells into a subject, and includes, for example, spontaneously arising cancer, cancer caused by exposure of a patient to a carcinogen(s), cancer resulting from insertion of a transgenic oncogene or knockout of a tumor suppressor gene, and cancer caused by infections, e.g., viral infections. The term “carcinoma” is art recognized and refers to malignancies of epithelial or endocrine tissues. The term also includes carcinosarcomas, which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation. The term “hematopoietic neoplastic disorders” includes diseases involving hyperplastic/neoplastic cells of hematopoietic origin. A hematopoietic neoplastic disorder can arise from myeloid, lymphoid or erythroid lineages, or precursor cells thereof. A hematologic cancer is a cancer that begins in blood-forming tissue, such as the bone marrow, or in the cells of the immune system. Examples of hematologic cancer include e.g., leukemia, lymphoma, and multiple myeloma etc.

As used herein, the terms “subject” and “patient” are used interchangeably throughout the specification and describe an animal, human or non-human, to whom treatment according to the methods of the present invention is provided. Veterinary and non-veterinary applications are contemplated in the present disclosure. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

As used herein, the terms “polypeptide,” “peptide,” and “protein” are used interchangeably to refer to polymers of amino acids of any length of at least two amino acids.

As used herein, the terms “polynucleotide,” “nucleic acid molecule,” and “nucleic acid sequence” are used interchangeably herein to refer to polymers of nucleotides of any length of at least two nucleotides, and include, without limitation, DNA, RNA, DNA/RNA hybrids, and modifications thereof.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1A shows a 3D structure and a corresponding 2D schematic diagram of wildtype IL-7 without a signal peptide (“Original IL-7”).

FIG. 1B shows a 3D structure and a corresponding 2D schematic diagram of rearranged IL-7 variants (“Designer IL-7”).

FIGS. 2A-2B show sequences of IL-7 variants and wildtype IL-7. Mutations and amino acids representing the artificial DA loop are underlined. For IL-7-wt (SEQ ID NO: 2), helices A-D are highlighted in bold.

FIG. 3 shows pSTAT5 phosphorylation ELISA assay results. OD450 was measured using serially diluted His-tagged IL-7 variants.

FIG. 4 shows primary T cell proliferation assay results. Luminescence was measured using serially diluted His-tagged IL-7 variants.

FIG. 5 shows ELISA binding assay results indicating binding capacities to human CD127. OD450 was measured using serially diluted His-tagged IL-7 variants.

FIG. 6 shows sequences of IL-7G variants. Mutations and amino acids representing the artificial DA loop are underlined.

FIG. 7 shows primary T cell proliferation assay results. Luminescence was measured using serially diluted His-tagged IL-7G variants.

FIG. 8 shows primary T cell proliferation assay results. Luminescence was measured using serially diluted hG4-IL-7G variants.

FIG. 9 shows ELISA binding assay results indicating binding capacities to human CD127. OD450 was measured using serially diluted hG4-IL-7 variants.

FIG. 10 lists additional sequences discussed in the disclosure.

FIG. 11A is a table showing treatment plan and dosing schedule of IgG4 Fc-IL-7 and its variants in a CT26-bearing BLAB/c mouse model.

FIG. 11B shows the average tumor growth curves of CT26-bearing BALB/c mice that were treated with IgG4 Fc-IL-7 and its variants. After inoculation (Day 0), the mice were injected with IgG4 Fc-IL-7 and its variants on Day 4 and Day 11 (indicated by arrows).

FIG. 11C show individual tumor growth curves of CT26-bearing BALB/c mice.

FIG. 11D shows individual tumor volume of each CT26-bearing BALB/c mouse that were treated with IgG4 Fc-IL-7 and its variants on Day 18 (18 days after inoculation).

### DETAILED DESCRIPTION

Interleukin-7 (IL-7) is a four-α-helical cytokine that binds to a receptor comprising IL-7Rα (CD127) and the common cytokine receptor γ chain (CD132). IL-7 is critical for development and maintenance of the entire lymphoid compartment, including T cells, B cells and ILCs. IL-7 promotes the survival and expansion of naive T cells or memory T cells, but not Treg cells. IL-7 can decrease PD-1 expression and restore functional capacity of exhausted T cells.

IL-7 is a cytokine essential for the adaptive immune system. T lymphopoiesis in the thymus has been shown to be highly IL-7 dependent in mice. In addition, IL-7 is important for T cell homeostasis and lymphopenia-driven proliferation. It regulates lymph nodes (LN) organogenesis by controlling the pool of lymphoid tissue inducer (LTi) cells. By activating several intracellular signal pathways, IL-7 promotes the cell survival and proliferation of both naïve and memory T cells. IL-7 is mainly produced by non-hematopoietic cells including keratinocytes in the skin, fibroblastic stromal cells in the bone marrow and lymphoid organs, epithelial cells in the thymus, prostatic epithelium and the intestine. Immune cells, such as dendritic cells (DCs) can also produce IL-7. Moreover, IL-7 transcripts and proteins have also been found in normal adult human hepatic tissue produced by cells of lymphoid morphology.

The receptor of IL-7 is a heterodimer that consists of two chains: IL-7Rα (CD127), which is shared with thymic stromal lymphopoietin (TSLP), and the common γ chain (CD132) for IL-2, IL-4, IL-9, IL-15 and IL-21. The γ chain is expressed on all hematopoietic cell types, while IL-7Rα is mainly expressed by lymphocytes, including common T/B lymphoid precursors, developing T and B cells, naïve T cells and memory T cells. Innate lymphoid cells (ILCs) are critical in lymphoid organ development and innate immune responses to pathogens. IL-7Rα is also found in ILCs, such as NK cells and gut-associated lymphoid tissue (GALT)-derived LTi cells. IL-7 can also regulate lymphoid organogenesis by controlling the pool of LTi cells. IL-7Rα is regulated by stimulative transcription factors, GABPα and Foxol as well as inhibitory Gfi-1. TGF-β promotes IL-7Rα expression via the inhibition of Gfi-1 expression. There is another type of IL-7 receptor: soluble IL-7R, which competes with cell-associated IL-7R to reduce excessive IL-7 consumption by IL-7R expressing target cells and enhances the bioactivity of IL-7 when the cytokine is limited.

There are two main signaling pathways responsible for the function of IL-7: Jak-Stat and PI3K-Akt. IL-7Rα is associated with the protein tyrosine kinase Janus kinase 1 (Jak1), and the cytosolic tail of the γ chain is associated with Jak3. Binding of IL-7 to its receptor causes activation of Jaks in the cytosol, phosphorylating signal transducer and activator of transcription (STAT) proteins. The dimeric phosphorylated STAT (pSTAT) proteins subsequently translocate into the nucleus to activate gene expression. Via the Jak3-Stat5 pathway, IL-7 activates the anti-apoptotic genes, Bcl-2 and Mcl-1, and suppresses pro-apoptotic proteins, such as Bax and Bak. Consequently, naïve and memory T cells survive. This function is dose-dependent, such that a higher concentration of IL-7 induces thymic emigrant T cell proliferation, while lower concentrations sustain cell survival. By activating the PI3K-Akt pathway, IL-7 downregulates the cell cycle inhibitor p27^{kipl} to induce the expression of cyclin D1 for cell cycle progression. Moreover, it promotes glucose transporter 1 expression, glucose uptake and mitochondrial integrity to positively regulate cell metabolism and size.

A detailed description of IL-7 and its function can be found, e.g., in Gao, J., et al. “Mechanism of action of IL-7 and its potential applications and limitations in cancer immunotherapy.” International Journal of Molecular Sciences 16.5 (2015): 10267-10280; Lin, J., et al. “The role of IL-7 in Immunity and Cancer.” Anticancer Research 37.3 (2017): 963-967; and McElroy, C. A., et al. “Structural and biophysical studies of the human IL-7/IL-7Rα complex.” Structure 17.1 (2009): 54-65; each of which is incorporated by reference herein in the entirety.

### Engineered IL-7 Variants

The human IL-7 gene locus is 72 kb in length, resides on chromosome 8q12-13 and encodes for a protein of 177 amino acids (SEQ ID NO: 1) with a molecular weight of 20 kDa. Human IL-7 (UniProt identifier: P13232) contains an N-terminal signal peptide that corresponds to amino acids 1-25 of SEQ ID NO: 1, and four α-helical domains (helices), i.e., helix A, helix B, helix C, and helix D. In a wildtype IL-7, helix A corresponds to amino acids 31-52 of SEQ ID NO: 1; helix B corresponds to amino acids 77-92 of SEQ ID NO: 1; helix C corresponds to amino acids 98-117 of SEQ ID NO: 1; and helix D corresponds to amino acids 152-172 of SEQ ID NO: 1. And the order of these helices in a wildtype IL-7 is helix A, helix B, helix C, and helix D from N-terminus to C-terminus.

The present disclosure provides engineered IL-7 polypeptides and engineered IL-7 variants. In some embodiments, the engineered IL-7 variants described herein are any of the engineered IL-7 polypeptides. In some embodiments, the engineered IL-7 polypeptide includes, from N-terminus to C-terminus, helix D, helix A, helix B, and helix C. As used herein, helix A corresponds to amino acids 31-52 of SEQ ID NO: 1 or the variant thereof; helix B corresponds to amino acids 77-92 of SEQ ID NO: 1 or the variant thereof; helix C corresponds to amino acids 98-117 of SEQ ID NO: 1 of the variant thereof; and helix D corresponds to amino acids 152-172 of SEQ ID NO: 1 or the variant thereof.

In some embodiments, the helix A includes a minimal sequence that is set forth in SEQ ID NO: 31. In some embodiments, the helix B includes a minimal sequence that is set forth in SEQ ID NO: 32. In some embodiments, the helix C includes a minimal sequence that is set forth in SEQ ID NO: 33. In some embodiments, the helix D includes a minimal sequence that is set forth in SEQ ID NO: 30. In some embodiments, the helix D comprises or consists of an amino acid sequence that is at least 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 100% to SEQ ID NO: 30. In some embodiments, the helix A comprises or consists of an amino acid sequence that is at least 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 100% to SEQ ID NO: 31. In some embodiments, the helix B comprises or consists of an amino acid sequence that is at least 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 100% to SEQ ID NO: 32. In some embodiments, the helix C comprises or consists of an amino acid sequence that is at least 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 100% to SEQ ID NO: 33. In some embodiments, the engineered IL-7 polypeptide includes a DA loop between helix D and helix A.

In some embodiments, the engineered IL-7 polypeptide includes, from N-terminus to C-terminus, a helix D fragment and a helix ABC fragment. The helix D fragment comprises helix D. The helix ABC fragment comprises helix A, helix B, and helix C from N-terminus to C-terminus. In some embodiments, the helix D fragment and the helix ABC fragment are linked via a DA loop. In some embodiments, the helix D fragment comprises or consists of an amino acid sequence that is at least 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 100% to SEQ ID NO: 50. In some embodiments, the helix ABC fragment comprises or consists of an amino acid sequence that is at least 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 100% to SEQ ID NO: 50 or SEQ ID NO: 52.

The DA loop sequence can be any appropriate linker sequence. In some embodiments, the DA loop described herein comprises or consists of an amino acid sequence that is at least 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOs: 34-49.

In some embodiments, the engineered IL-7 polypeptide comprises or consists of an amino acid sequence that is at least 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 1, 2, 3, 4, 5, 6, 7, 8, 9, 10, 11, 12, 13, 14, 15, 16, 17, 18, 19, 21, 22, 23, 24, 25, 26, or 27.

In some embodiments, the engineered IL-7 polypeptide comprises or consists of an amino acid sequence that is at least 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 21. In some embodiments, the engineered IL-7 polypeptide comprises or consists of an amino acid sequence that is at least 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 23. In some embodiments, the engineered IL-7 polypeptide comprises or consists of an amino acid sequence that is at least 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 25. In some embodiments, the engineered IL-7 polypeptide comprises or consists of an amino acid sequence that is at least 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 27.

In some embodiments, the engineered IL-7 variants can have at least or about 1 (e.g., at least or about 2, at least or about 3, at least or about 4, at least or about 5, at least or about 6, at least or about 7, at least or about 8, at least or about 9, at least or about 10) amino acid insertions, deletions, or substitutions as compared to any one of SEQ ID NOs: 1-19 and 21-27.

In some embodiments, the engineered IL-7 polypeptide has one or more of the following mutations:
(a) the amino acid that corresponds to N95 of SEQ ID NO: 1 is Q, E, or A;
(b) the amino acid that corresponds to N116 of SEQ ID NO: 1 is Q, E, or A; and/or
(c) the amino acid that corresponds to N141 of SEQ ID NO: 1 is Q, E, or A.

In some embodiments, the engineered IL-7 polypeptide comprises the helix D fragment and/or the helix ABC fragment described herein.

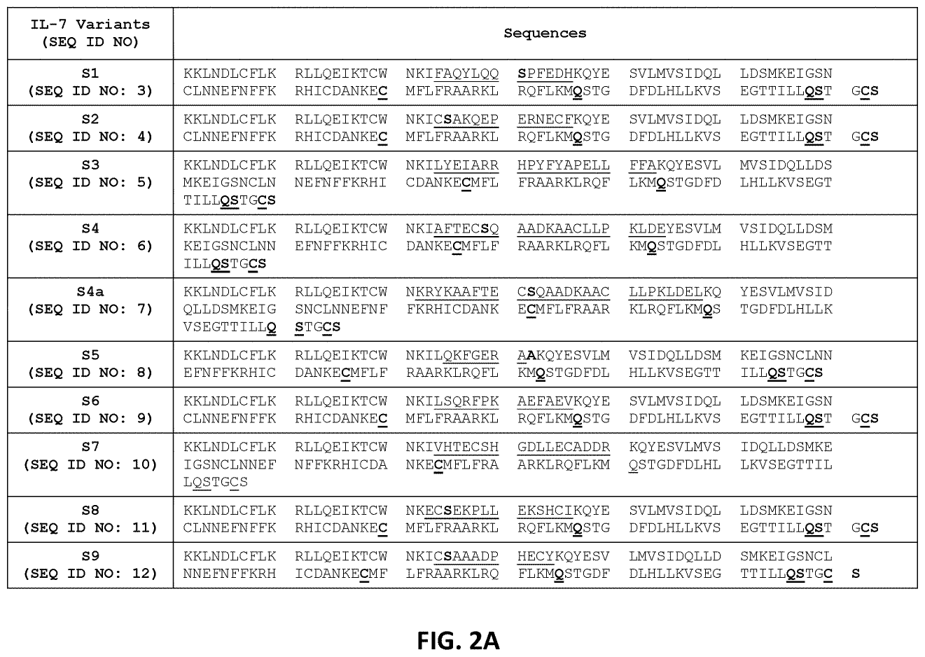

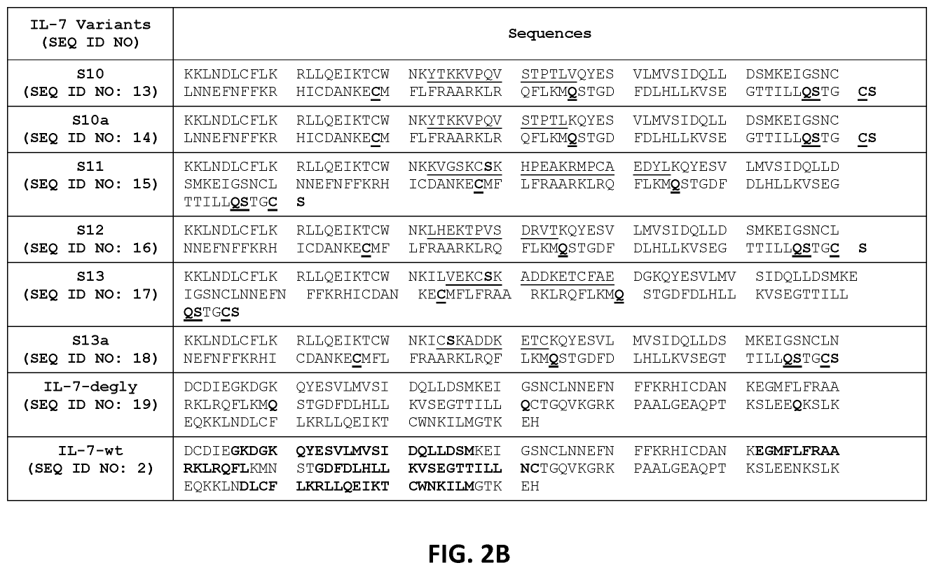

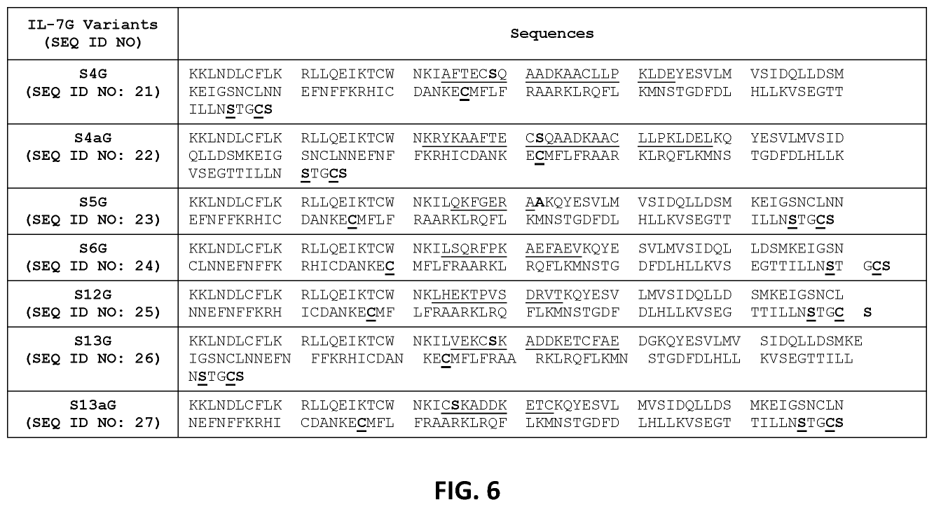

In some embodiments, the engineered IL-7 polypeptide comprises or consists of an amino acid sequence that is at least 80%, 85%, 90%, or 95% identical to any one of SEQ ID NOs: 2-19 and 21-27 with 0, 1, 2, 3, 4, 5, 6, 7, 8, 9, 10, 11, 12, 13, 14, or 15 mutations as shown in FIGS. 2A-2B and FIG. 6.

The engineered IL-7 polypeptide can have additional modifications. In some embodiments, the engineered IL-7 polypeptide can have a CH2 domain and/or a CH3 domain of Fc. In some embodiments, the engineered IL-7 polypeptide can be linked to the N-terminal of the CH2 domain (e.g., through an optional hinge region or a GS linker). In some embodiments, the engineered IL-7 polypeptide can be linked to the C-terminal of the CH3 domain (e.g., through an optional GS linker). In some embodiments, the hinge region is an IgG hinge region (e.g., IgG4 hinge region). In some embodiments, the CH2 domain is an IgG CH2 domain (e.g., IgG4 CH2 domain). In some embodiments, the CH3 domain is an IgG CH3 domain (e.g., IgG4 CH3 domain). In some embodiments, the hinge region, the CH2 domain, the CH3 domain have a sequence that is at least 80%, 85%, 90%, 95%, 100% identical to SEQ ID NO: 28.

In some embodiments, the engineered IL7 polypeptide described herein further includes a His-tag. In some embodiments, the His-tag includes an amino acid sequence that is at least 80%, 85%, 90%, or 95% identical to SEQ ID NO: 20.

### IL-7 Protein Constructs

The disclosure provides engineered IL-7 protein constructs that can specifically bind to CD127 (e.g., human CD127) and/or CD132 (e.g., human CD132). In some embodiments, these protein constructs can regulate development and maintenance of entire lymphoid compartment (including T cells, B cells, and ILCs); promote survival and expansion of naïve T cells or memory T cells; decrease PD-1 expression; restore functional capacity of exhausted T cells; enhance T cell survival and proliferation; decrease exhausted TILs in tumor microenvironment; induce STAT5 phosphorylation on primary T cells; compete with endogenous IL-7 and block IL-7/IL-7R interaction; and/or induce primary T cell proliferation.

In some embodiments, the engineered IL-7 protein constructs can comprise any engineered IL-7 variant as described herein. In some embodiments, the engineered IL-7 protein constructs can have a sequence that is at least 80%, 81%, 82%, 83%, 84%, 85%, 86%, 87%, 88%, 89% 90%, 91%, 92%, 93%, 94%, 95%, 96%, 97%, 98%, 99% or 100% identical to any sequence of SEQ ID NOs: 1-19 and 21-27. In some embodiments, the engineered IL-7 protein constructs can comprise or consists of a sequence that is at least 80%, 81%, 82%, 83%, 84%, 85%, 86%, 87%, 88%, 89% 90%, 91%, 92%, 93%, 94%, 95%, 96%, 97%, 98%, 99% or 100% identical to any sequence of SEQ ID NOs: 50, 51, and/or 52.

The disclosure also provides nucleic acid comprising a polynucleotide encoding a polypeptide comprising a sequence that is at least 80%, 81%, 82%, 83%, 84%, 85%, 86%, 87%, 88%, 89% 90%, 91%, 92%, 93%, 94%, 95%, 96%, 97%, 98%, 99% or 100% identical to any sequence selected from the group consisting of SEQ ID NOs: 1-19 and 21-27 or SEQ ID NOs: 50, 51, and 52.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For example, the comparison of sequences and determination of percent identity between two sequences can be accomplished using a Blossum 62 scoring matrix with a gap penalty of 12, a gap extend penalty of 4, and a frameshift gap penalty of 5.

The engineered IL-7 protein constructs can further comprises an Fc region of an antibody. These antibodies can be of any type (e.g., IgG, IgE, IgM, IgD, IgA, and IgY), class or subclass (e.g., IgG1, IgG2, IgG3, IgG4, IgA1, IgA2, IgE1, IgE2). In some embodiments, the he Fc region is derived from human IgG (e.g., IgG1, IgG2, IgG3, or IgG4). In some embodiments, the Fc region is an IgG4 Fc region (e.g., human IgG4 Fc region).

In some embodiments, the engineered IL-7 variant is linked to the Fc region through a linker sequence (e.g., an antibody hinge region such as IgG, IgE hinge region). In addition, the Fc region can be modified to provide desired effector functions or serum half-life.

In some embodiments, the engineered IL-7 variant is linked to the N terminus of the Fc region. In some embodiments, the engineered IL-7 variant is linked to the C terminus of the Fc region.

In some embodiments, the engineered IL-7 variants and protein constructs as described herein can increase immune response, activity or number of immune cells (e.g., primary T cells) by at least 10%, 20%, 30%, 40%, 50%, 60%, 70%, 80%, 90%, 100%, 2 folds, 3 folds, 5 folds, 10 folds, or 20 folds.

In some implementations, the engineered IL-7 variants and protein constructs can bind to IL-7 (e.g., human IL-7, monkey IL-7 (e.g., cynomolgus monkey (Macaca fascicularis), mouse IL-7) with a dissociation rate (k_{off}) of less than 0.1 s⁻¹, less than 0.01 s⁻¹, less than 0.001 s⁻¹, less than 0.0001 s⁻¹, or less than 0.00001 s⁻¹. In some embodiments, the dissociation rate (k_{off}) is greater than 0.01 s⁻¹, greater than 0.001 s⁻¹, greater than 0.0001 s⁻¹, greater than 0.00001 s⁻¹, or greater than 0.000001 s⁻¹.

In some embodiments, kinetic association rates (kₒₙ) is greater than 1×10²/Ms, greater than 1×10³/Ms, greater than 1×10⁴/Ms, greater than 1×10⁵/Ms, or greater than 1×10⁶/Ms. In some embodiments, kinetic association rates (kₒₙ) is less than 1×10⁵/Ms, less than 1×10⁶/Ms, or less than 1×10⁷/Ms.

Affinities can be deduced from the quotient of the kinetic rate constants (KD=K_{off}/kₒₙ). In some embodiments, KD is less than 1×10⁻⁶ M, less than 1×10⁻⁷M, less than 1×10⁻⁸ M, less than 1×10⁻⁹ M, or less than 1×10⁻¹⁰ M. In some embodiments, the KD is less than 300 nM, 200 nM, 100 nM, 50 nM, 30 nM, 20 nM, 15 nM, 10 nM, 9 nM, 8 nM, 7 nM, 6 nM, 5 nM, 4 nM, 3 nM, 2 nM, 1 nM, 900 pM, 800 pM, 700 pM, 600 pM, 500 pM, 400 pM, 300 pM, 200 pM, 100 pM, 90 pM, 80 pM, 70 pM, 60 pM, 50 pM, 40 pM, 30 pM, 20 pM, or 10 pM. In some embodiments, KD is greater than 1×10⁻⁷M, greater than 1×10⁻⁸M, greater than 1×10⁻⁹ M, greater than 1×10⁻¹⁰ M, greater than 1×10⁻¹¹ M, or greater than 1×10⁻¹² M.

General techniques for measuring the affinity include, e.g., ELISA, RIA, and surface plasmon resonance (SPR). In some embodiments, the engineered IL-7 variants and protein constructs can bind to monkey IL-7, and/or mouse IL-7. In some embodiments, the engineered IL-7 variants and protein constructs cannot bind to monkey IL-7, and/or mouse IL-7.

In some embodiments, the engineered IL-7 variants and/or protein constructs as described herein has a tumor growth inhibition percentage (TGI %) that is greater than 10%, 20%, 30%, 40%, 50%, 60%, 70%, 80%, 90%, 100%, 110%, 120%, 130%, 140%, 150%, 160%, 170%, 180%, 190%, or 200%. In some embodiments, the engineered IL-7 variants and/or protein constructs as described herein has a tumor growth inhibition percentage that is less than 60%, 70%, 80%, 90%, 100%, 110%, 120%, 130%, 140%, 150%, 160%, 170%, 180%, 190%, or 200%. The TGI % can be determined, e.g., at 3, 4, 5, 6, 7, 8, 9, 10, 11, 12, 13, 14, 15, 16, 17, 18, 19, 20, 21, 22, 23, 24, 25, 26, 27, 28, 29, or 30 days after the treatment starts, or 1, 2, 3, 4, 5, 6, 7, 8, 9, 10, 11, or 12 months after the treatment starts. As used herein, the tumor growth inhibition percentage (TGI %) is calculated using the following formula:

$$TGI ( \% ) = [ 1 - ( Ti - T 0 ) / ( Vi - V 0 ) ] \times 100$$

Ti is the average tumor volume in the treatment group on day i. T0 is the average tumor volume in the treatment group on day zero. Vi is the average tumor volume in the control group on day i. V0 is the average tumor volume in the control group on day zero. In some embodiments, the engineered IL-7 variants and/or protein constructs thereof as described herein can inhibit tumor growth.

In some embodiments, the protein constructs as described herein have a functional Fc region. In some embodiments, the Fc region is human IgG1, human IgG2, human IgG3, or human IgG4. In some embodiments, effector function of a functional Fc region is antibody-dependent cell-mediated cytotoxicity (ADCC). In some embodiments, effector function of a functional Fc region is phagocytosis. In some embodiments, effector function of a functional Fc region is ADCC and phagocytosis. In some embodiments, the protein constructs as described herein have an Fc region without effector function. In some embodiments, the Fc is a human IgG4 Fc. In some embodiments, the Fc does not have a functional Fc region. For example, the Fc region has LALA mutations (L234A and L235A mutations in EU numbering), or LALA-PG mutations (L234A, L235A, P329G mutations in EU numbering).

Some other modifications to the Fc region can be made. For example, a cysteine residue(s) can be introduced into the Fc region, thereby allowing interchain disulfide bond formation in this region. The homodimeric fusion protein thus generated may have any increased half-life in vitro and/or in vivo.

In some embodiments, the IgG4 has S228P mutation (EU numbering). The S228P mutation prevents in vivo and in vitro IgG4 Fab-arm exchange.

In some embodiments, Fc regions are provided having a carbohydrate structure that lacks fucose attached (directly or indirectly) to an Fc region. For example, the amount of fucose in such Fc region composition may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues; or position 314 in Kabat numbering); however, Asn297 may also be located about ±3 amino acids upstream or downstream of position 297, i.e., between positions 294 and 300, due to minor sequence variations in Fc region sequences. Such fucosylation variants may have improved ADCC function. In some embodiments, to reduce glycan heterogeneity, the Fc region can be further engineered to replace the Asparagine at position 297 with Alanine (N297A).

In some embodiments, the binding affinity between CD127 (e.g., human CD127, monkey CD127, mouse CD127, or extracellular domains thereof) and the engineered IL-7 variants and/or protein constructs as described herein is at least 1-fold, 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, or 50-fold as compared to that between CD127 and a wild-type IL-7 or protein constructs thereof.

In some embodiments, the main peak of HPLC-SEC accounts for at least 90%, 95%, 96%, 97%, 98%, 99%, or 99.5% of the engineered IL-7 variants and/or protein constructs as described herein after purification by a protein A column.

### Methods of Making Engineered IL-7 Variants and Protein Constructs

Variants of the IL-7 described herein can be prepared by introducing appropriate nucleotide changes into the DNA encoding a IL-7 peptide or a part thereof or by peptide synthesis. Such variants include, for example, deletions, insertions, or substitutions of residues within the amino acids sequences.

Screening can be performed. In a population of such variants, some engineered IL-7 variants will have increased affinity for the CD127. Any combination of deletions, insertions, and/or combinations can be made to arrive at a variant that has increased binding affinity for the target. The amino acid changes introduced into the variant can also alter or introduce new post-translational modifications into the polypeptide, such as changing (e.g., increasing or decreasing) the number of glycosylation sites, changing the type of glycosylation site (e.g., changing the amino acid sequence such that a different sugar is attached by enzymes present in a cell), or introducing new glycosylation sites.

Engineered IL-7 variants can be derived from any species of animal, including mammals. Non-limiting examples of IL-7 variants include IL-7 variants derived from humans, primates, e.g., monkeys and apes, cows, pigs, horses, sheep, camelids (e.g., camels and llamas), chicken, goats, and rodents (e.g., rats, mice, hamsters and rabbits).

The present disclosure also provides recombinant vectors (e.g., an expression vectors) that include an isolated polynucleotide disclosed herein (e.g., a polynucleotide that encodes a polypeptide disclosed herein), host cells into which are introduced the recombinant vectors (i.e., such that the host cells contain the polynucleotide and/or a vector comprising the polynucleotide), and the production of recombinant polypeptides or fragments thereof by recombinant techniques.

As used herein, a “vector” is any construct capable of delivering one or more polynucleotide(s) of interest to a host cell when the vector is introduced to the host cell. An “expression vector” is capable of delivering and expressing the one or more polynucleotide(s) of interest as an encoded polypeptide in a host cell into which the expression vector has been introduced. Thus, in an expression vector, the polynucleotide of interest is positioned for expression in the vector by being operably linked with regulatory elements such as a promoter, enhancer, and/or a poly-A tail, either within the vector or in the genome of the host cell at or near or flanking the integration site of the polynucleotide of interest such that the polynucleotide of interest will be translated in the host cell introduced with the expression vector.

A vector can be introduced into the host cell by methods known in the art, e.g., electroporation, chemical transfection (e.g., DEAE-dextran), transformation, transfection, and infection and/or transduction (e.g., with recombinant virus). Thus, non-limiting examples of vectors include viral vectors (which can be used to generate recombinant virus), naked DNA or RNA, plasmids, cosmids, phage vectors, and DNA or RNA expression vectors associated with cationic condensing agents.

In some implementations, a polynucleotide disclosed herein (e.g., a polynucleotide that encodes a polypeptide disclosed herein) is introduced using a viral expression system (e.g., vaccinia or other pox virus, retrovirus, or adenovirus), which may involve the use of a non-pathogenic (defective), replication competent virus, or may use a replication defective virus. Techniques for incorporating DNA into such expression systems are well known to those of ordinary skill in the art. The DNA may also be “naked.” The uptake of naked DNA may be increased by coating the DNA onto biodegradable beads that are efficiently transported into the cells.

For expression, the DNA insert comprising a polypeptide-encoding polynucleotide disclosed herein can be operatively linked to an appropriate promoter (e.g., a heterologous promoter), such as the phage lambda PL promoter, the E. coli lac, trp and tac promoters, the SV40 early and late promoters and promoters of retroviral LTRs, to name a few. Other suitable promoters are known to the skilled artisan. In some embodiments, the promoter is a cytomegalovirus (CMV) promoter. The expression constructs can further contain sites for transcription initiation, termination and, in the transcribed region, a ribosome binding site for translation. The coding portion of the mature transcripts expressed by the constructs may include a translation initiating at the beginning and a termination codon (UAA, UGA, or UAG) appropriately positioned at the end of the polypeptide to be translated.

As indicated, the expression vectors can include at least one selectable marker. Such markers include dihydrofolate reductase or neomycin resistance for eukaryotic cell culture and tetracycline or ampicillin resistance genes for culturing in E. coli and other bacteria. Representative examples of appropriate hosts include, but are not limited to, bacterial cells, such as E. coli, Streptomyces, and Salmonella typhimurium cells; fungal cells, such as yeast cells; insect cells such as Drosophila S2 and Spodoptera Sf9 cells; animal cells such as CHO, COS, Bowes melanoma, and HK 293 cells; and plant cells. Appropriate culture mediums and conditions for the host cells described herein are known in the art.

Non-limiting vectors for use in bacteria include pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Non-limiting eukaryotic vectors include pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Non-limiting bacterial promoters suitable for use include the E. coli lacI and lacZ promoters, the T3 and T7 promoters, the gpt promoter, the lambda PR and PL promoters and the trp promoter. Suitable eukaryotic promoters include the CMV immediate early promoter, the HSV thymidine kinase promoter, the early and late SV40 promoters, the promoters of retroviral LTRs, such as those of the Rous sarcoma virus (RSV), and metallothionein promoters, such as the mouse metallothionein-I promoter.

In the yeast Saccharomyces cerevisiae, a number of vectors containing constitutive or inducible promoters such as alpha factor, alcohol oxidase, and PGH can be used.

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986), which is incorporated herein by reference in its entirety.

Transcription of DNA encoding a polypeptide of the present disclosure by higher eukaryotes may be increased by inserting an enhancer sequence into the vector. Enhancers are cis-acting elements of DNA, usually about from 10 to 300 bp that act to increase transcriptional activity of a promoter in a given host cell-type. Examples of enhancers include the SV40 enhancer, which is located on the late side of the replication origin at base pairs 100 to 270, the cytomegalovirus early promoter enhancer, the polyoma enhancer on the late side of the replication origin, and adenovirus enhancers.

For secretion of the translated protein into the lumen of the endoplasmic reticulum, into the periplasmic space or into the extracellular environment, appropriate secretion signals may be incorporated into the expressed polypeptide. The signals may be endogenous to the polypeptide or they may be heterologous signals.

The polypeptide (e.g., IL-7 variants) can be expressed in a modified form, such as a fusion protein (e.g., a GST-fusion) or with a histidine-tag, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties can be added to the polypeptide to facilitate purification. Such regions can be removed prior to final preparation of the polypeptide. The addition of peptide moieties to polypeptides to engender secretion or excretion, to improve stability and to facilitate purification, among others, are familiar and routine techniques in the art.

In some embodiments, the engineered IL-7 variants or protein constructs described herein do not include an IL-7 signal peptide, e.g., a sequence corresponding to amino acids 1-25 of SEQ ID NO: 1.

### Methods of Treatment

The engineered IL-7 variants and protein constructs of the present disclosure can be used for various therapeutic purposes.

In one aspect, the disclosure provides methods for treating a cancer in a subject, methods of reducing the rate of the increase of volume of a tumor in a subject over time, methods of reducing the risk of developing a metastasis, or methods of reducing the risk of developing an additional metastasis in a subject. In some embodiments, the treatment can halt, slow, retard, or inhibit progression of a cancer. In some embodiments, the treatment can result in the reduction of in the number, severity, and/or duration of one or more symptoms of the cancer in a subject.

In one aspect, the disclosure features methods that include administering a therapeutically effective amount of engineered IL-7 variants and protein constructs disclosed herein to a subject in need thereof (e.g., a subject having, or identified or diagnosed as having, a cancer), e.g., breast cancer (e.g., triple-negative breast cancer), carcinoid cancer, cervical cancer, endometrial cancer, glioma, head and neck cancer, liver cancer, lung cancer, small cell lung cancer, lymphoma, melanoma, ovarian cancer, pancreatic cancer, prostate cancer, renal cancer, colorectal cancer, gastric cancer, testicular cancer, thyroid cancer, bladder cancer, urethral cancer, stomach cancer, urothelial cancer, skin cancer, or hematologic malignancy. In some embodiments, the cancer is unresectable melanoma or metastatic melanoma, non-small cell lung carcinoma (NSCLC), small cell lung cancer (SCLC), bladder cancer, or metastatic hormone-refractory prostate cancer. In some embodiments, the subject has a solid tumor. In some embodiments, the cancer is squamous cell carcinoma of the head and neck (SCCHN), renal cell carcinoma (RCC), triple-negative breast cancer (TNBC), or colorectal carcinoma. In some embodiments, the cancer is melanoma, pancreatic carcinoma, mesothelioma, hematological malignancies, especially Non-Hodgkin's lymphoma, lymphoma, chronic lymphocytic leukemia, or advanced solid tumors. In some embodiments, the cancer is lung cancer, melanoma, colorectal cancer, glioma, or pancreatic cancer.

In some embodiments, the compositions and methods disclosed herein can be used for treatment of patients at risk for a cancer. Patients with cancer can be identified with various methods known in the art.

Because of the IL-7 function to increase lymphocyte survival and number, in some embodiments, the compositions and methods described herein can be used for treatment of lymphopenia and/or infectious diseases.

As used herein, by an “effective amount” is meant an amount or dosage sufficient to effect beneficial or desired results including halting, slowing, retarding, or inhibiting progression of a disease, e.g., a cancer. An effective amount will vary depending upon, e.g., an age and a body weight of a subject to which the engineered IL-7 variants and protein constructs, vector comprising the polynucleotide encoding the engineered IL-7 variants and protein constructs, and/or compositions thereof is to be administered, a severity of symptoms and a route of administration, and thus administration can be determined on an individual basis.

An effective amount can be administered in one or more administrations. By way of example, an effective amount of the engineered IL-7 variants and/or protein constructs is an amount sufficient to ameliorate, stop, stabilize, reverse, inhibit, slow and/or delay progression of a cancer in a patient or is an amount sufficient to ameliorate, stop, stabilize, reverse, slow and/or delay proliferation of a cell (e.g., a biopsied cell, any of the cancer cells described herein, or cell line (e.g., a cancer cell line)) in vitro. As is understood in the art, an effective amount may vary, depending on, inter alia, patient history as well as other factors such as the type (and/or dosage) of the engineered IL-7 variants and protein constructs used.

Effective amounts and schedules for administering the engineered IL-7 variants and protein constructs, the polynucleotides encoding the engineered IL-7 variants and protein constructs, and/or compositions disclosed herein may be determined empirically, and making such determinations is within the skill in the art. Those skilled in the art will understand that the dosage that must be administered will vary depending on, for example, the mammal that will receive the engineered IL-7 variants and protein constructs, the polynucleotides, and/or compositions disclosed herein, the route of administration, the particular type of polynucleotides, and/or compositions disclosed herein used and other drugs being administered to the mammal.

A typical daily dosage of an effective amount of the engineered IL-7 variants and/or protein constructs is 0.1 mg/kg to 100 mg/kg (mg per kg of patient weight). In some embodiments, the dosage can be less than 100 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, or 0.1 mg/kg. In some embodiments, the dosage can be greater than 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, or 0.1 mg/kg. In some embodiments, the dosage is about 10 mg/kg, 9 mg/kg, 8 mg/kg, 7 mg/kg, 6 mg/kg, 5 mg/kg, 4 mg/kg, 3 mg/kg, 2 mg/kg, or 1 mg/kg. In some embodiments, the dosage is about 1 to 10 mg/kg, about 1 to 5 mg/kg, or about 2 to 5 mg/kg.

In any of the methods described herein, the engineered IL-7 variants and protein constructs can be administered to the subject at least once a week (e.g., once a week, twice a week, three times a week, four times a week, once a day, twice a day, or three times a day).

In some embodiments, the one or more additional therapeutic agents can be administered to the subject prior to, or after administering the engineered IL-7 variants and protein constructs. In some embodiments, the one or more additional therapeutic agents are administered to the subject such that there is an overlap in the bioactive period of the one or more additional therapeutic agents and the engineered IL-7 variants and protein constructs in the subject.

In some embodiments, one or more additional therapeutic agents can be administered to the subject. The additional therapeutic agent can comprise one or more inhibitors selected from the group consisting of an inhibitor of B-Raf, an EGFR inhibitor, an inhibitor of a MEK, an inhibitor of ERK, an inhibitor of K-Ras, an inhibitor of c-Met, an inhibitor of anaplastic lymphoma kinase (ALK), an inhibitor of a phosphatidylinositol 3-kinase (PI3K), an inhibitor of an Akt, an inhibitor of mTOR, a dual PI3K/mTOR inhibitor, an inhibitor of Bruton's tyrosine kinase (BTK), and an inhibitor of Isocitrate dehydrogenase 1 (IDH1) and/or Isocitrate dehydrogenase 2 (IDH2). In some embodiments, the additional therapeutic agent is an inhibitor of indoleamine 2,3-dioxygenase-1) (IDO1) (e.g., epacadostat).

In some embodiments, the additional therapeutic agent can comprise one or more inhibitors selected from the group consisting of an inhibitor of HER3, an inhibitor of LSD1, an inhibitor of MDM2, an inhibitor of BCL2, an inhibitor of CHK1, an inhibitor of activated hedgehog signaling pathway, and an agent that selectively degrades the estrogen receptor.

In some embodiments, the additional therapeutic agent can comprise one or more therapeutic agents selected from the group consisting of Trabectedin, nab-paclitaxel, Trebananib, Pazopanib, Cediranib, Palbociclib, everolimus, fluoropyrimidine, IFL, regorafenib, Reolysin, Alimta, Zykadia, Sutent, temsirolimus, axitinib, everolimus, sorafenib, Votrient, Pazopanib, IMA-901, AGS-003, cabozantinib, Vinflunine, an Hsp90 inhibitor, Ad-GM-CSF, Temazolomide, IL-2, IFNa, vinblastine, Thalomid, dacarbazine, cyclophosphamide, lenalidomide, azacytidine, lenalidomide, bortezomid, amrubicine, carfilzomib, pralatrexate, and enzastaurin.

In some embodiments, the additional therapeutic agent can comprise one or more therapeutic agents selected from the group consisting of an adjuvant, a TLR agonist, tumor necrosis factor (TNF) alpha, IL-1, HMGB1, an IL-10 antagonist, an IL-4 antagonist, an IL-13 antagonist, an IL-17 antagonist, an HVEM antagonist, an ICOS agonist, a treatment targeting CX3CL1, a treatment targeting CXCL9, a treatment targeting CXCL10, a treatment targeting CCL5, an LFA-1 agonist, an ICAM1 agonist, and a Selectin agonist.

In some embodiments, carboplatin, nab-paclitaxel, paclitaxel, cisplatin, pemetrexed, gemcitabine, FOLFOX, or FOLFIRI are administered to the subject.

In some embodiments, the additional therapeutic agent is an anti-OX40 antibody, an anti-PD-1 antibody, an anti-PD-L1 antibody, an anti-PD-L2 antibody, an anti-LAG-3 antibody, an anti-TIGIT antibody, an anti-BTLA antibody, an anti-CTLA-4 antibody, or an anti-GITR antibody. In some embodiments, the additional therapeutic agent is an anti-CD20 antibody (e.g., rituximab) or an anti-EGF receptor antibody (e.g., cetuximab).

### Pharmaceutical Compositions and Routes of Administration

Also provided herein are pharmaceutical compositions that contain the engineered IL-7 variants and protein constructs described herein. The pharmaceutical compositions can be formulated in any manner known in the art.

Pharmaceutical compositions are formulated to be compatible with their intended route of administration (e.g., intravenous, intraarterial, intramuscular, intradermal, subcutaneous, or intraperitoneal). The compositions can include a sterile diluent (e.g., sterile water or saline), a fixed oil, polyethylene glycol, glycerine, propylene glycol or other synthetic solvents, antibacterial or antifungal agents, such as benzyl alcohol or methyl parabens, chlorobutanol, phenol, ascorbic acid, thimerosal, and the like, antioxidants, such as ascorbic acid or sodium bisulfite, chelating agents, such as ethylenediaminetetraacetic acid, buffers, such as acetates, citrates, or phosphates, and isotonic agents, such as sugars (e.g., dextrose), polyalcohols (e.g., mannitol or sorbitol), or salts (e.g., sodium chloride), or any combination thereof. Liposomal suspensions can also be used as pharmaceutically acceptable carriers. Preparations of the compositions can be formulated and enclosed in ampules, disposable syringes, or multiple dose vials. Where required (as in, for example, injectable formulations), proper fluidity can be maintained by, for example, the use of a coating, such as lecithin, or a surfactant. Absorption of the agents can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid).

Compositions containing the engineered IL-7 variants and protein constructs described herein can be formulated for parenteral (e.g., intravenous, intraarterial, intramuscular, intradermal, subcutaneous, or intraperitoneal) administration in dosage unit form (i.e., physically discrete units containing a predetermined quantity of active compound for ease of administration and uniformity of dosage).

Pharmaceutical compositions for parenteral administration are preferably sterile and substantially isotonic and manufactured under Good Manufacturing Practice (GMP) conditions. Pharmaceutical compositions can be provided in unit dosage form (i.e., the dosage for a single administration). Pharmaceutical compositions can be formulated using one or more physiologically acceptable carriers, diluents, excipients or auxiliaries. The formulation depends on the route of administration chosen. For injection, the engineered IL-7 variants and protein constructs can be formulated in aqueous solutions, preferably in physiologically-compatible buffers to reduce discomfort at the site of injection. The solution can contain formulatory agents such as suspending, stabilizing and/or dispersing agents. Alternatively the engineered IL-7 variants and protein constructs can be in lyophilized form for constitution with a suitable vehicle, e.g., sterile pyrogen-free water, before use.

Toxicity and therapeutic efficacy of compositions can be determined by standard pharmaceutical procedures in cell cultures or experimental animals (e.g., monkeys). One can, for example, determine the LD50 (the dose lethal to 50% of the population) and the ED50 (the dose therapeutically effective in 50% of the population): the therapeutic index being the ratio of LD50:ED50. Agents that exhibit high therapeutic indices are preferred. Where an agent exhibits an undesirable side effect, care should be taken to minimize potential damage (i.e., reduce unwanted side effects). Toxicity and therapeutic efficacy can be determined by other standard pharmaceutical procedures.

Exemplary doses include milligram or microgram amounts of any of the engineered IL-7 variants and protein constructs described herein per kilogram of the subject's weight (e.g., about 1 μg/kg to about 500 mg/kg; about 100 μg/kg to about 500 mg/kg; about 100 μg/kg to about 50 mg/kg; about 10 μg/kg to about 5 mg/kg; about 10 μg/kg to about 0.5 mg/kg; about 1 μg/kg to about 50 μg/kg; about 1 mg/kg to about 10 mg/kg; or about 1 mg/kg to about 5 mg/kg). While these doses cover a broad range, one of ordinary skill in the art will understand that therapeutic agents can vary in their potency, and effective amounts can be determined by methods known in the art. Typically, relatively low doses are administered at first, and the attending health care professional or veterinary professional (in the case of therapeutic application) or a researcher (when still working at the development stage) can subsequently and gradually increase the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, and the half-life of the engineered IL-7 variants and protein constructs in vivo.

The pharmaceutical compositions can be included in a container, pack, or dispenser together with instructions for administration. The disclosure also provides methods of manufacturing the engineered IL-7 variants and protein constructs for various uses as described herein.

### Examples

The invention is further described in the following examples, which do not limit the scope of the invention described in the claims.

Example 1. Design of IL-7 Variants with Rearranged α-Helical Domains (Helices)

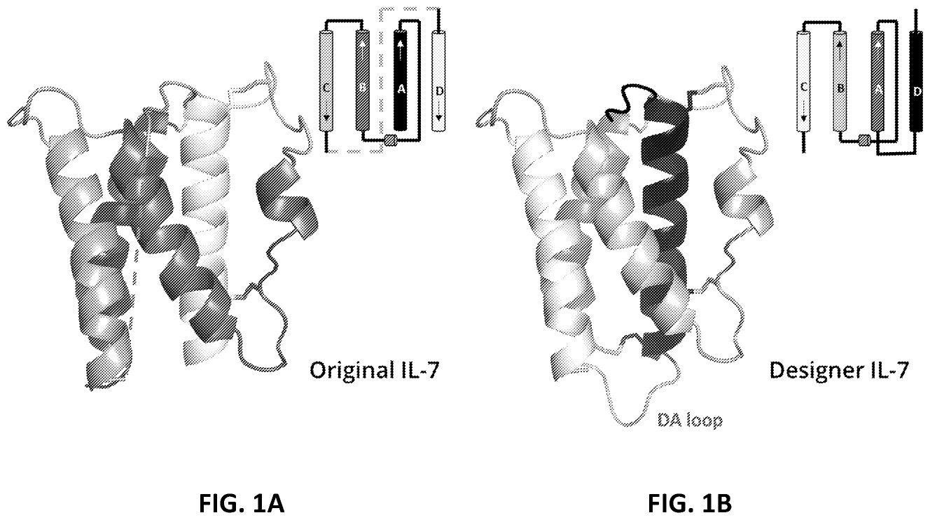

Human IL-7 (SEQ ID NO: 1) is a 177 amino acid cytokine that includes four α-helical domains (helices), from N-terminus to C-terminus: helix A, helix B, helix C, and helix D. The protein includes a signal peptide that corresponds to amino acids 1-25 of SEQ ID NO: 1. The mature IL-7 protein has an amino acid sequence set forth in SEQ ID NO: 2. A 3D structure and a 2D schematic diagram showing the helix arrangement of the wildtype IL-7 protein without a signal peptide (“original IL-7”) are shown in FIG. 1A.

To identify IL-7 variants with different functional potencies and higher structural stability, the four α-helical domains (helices) of the mature IL-7 protein was rearranged. As shown in FIG. 1B, helix D was rearranged from the C-terminus to the N-terminus of the protein. As a result, the rearranged IL-7 variants include, from N-terminus to C-terminus, helix D, helix A, helix B, and helix C. An artificial DA loop was also designed to connect the helix D and helix A.

Additional modifications (e.g., mutations) were introduced to further stabilize the rearranged IL-7 variants. For example, one or more of the three glycosylation sites corresponding to asparagine (Asn, or N) residues at positions 95, 116, and 141 of SEQ ID NO: 1 were also mutated to glutamine (Gln, or Q). The following IL-7 variants were obtained: S1 (SEQ ID NO: 3), S2 (SEQ ID NO: 4), S3 (SEQ ID NO: 5), S4 (SEQ ID NO: 6), S4a (SEQ ID NO: 7), S5 (SEQ ID NO: 8), S6 (SEQ ID NO: 9), S7 (SEQ ID NO: 10), S8 (SEQ ID NO: 11), S9 (SEQ ID NO: 12), S10 (SEQ ID NO: 13), S10a (SEQ ID NO: 14), S11 (SEQ ID NO: 15), S12 (SEQ ID NO: 16), S13 (SEQ ID NO: 17), and S13a (SEQ ID NO: 18). IL-7-degly (SEQ ID NO: 19) is a deglycosylated IL-7 variant without helix rearrangement. Sequences of the IL-7 variants are shown in the FIGS. 2A-2B.

The IL-7 variants were screened using a selection of assays. Specifically, reporter signaling assays (e.g., in HEK293 cells) and pSTAT5 assays (e.g., in primary T cells using ELISA kits) can be used to screen IL-7 variants with similar or slightly reduced signaling than wildtype IL-7; in vitro proliferation assays (e.g., in primary T/CD127-Jurkat cells) can be used to screen IL-7 variants with similar or slightly reduced signaling than wildtype IL-7, and exclude those with a high Treg cell expanding capability; and in vivo efficacy studies (e.g., in mouse tumor models) can be used to screen IL-7 variants with an anti-tumor response better than wildtype IL-7 and with lower toxicity.

### Example 2. In Vitro Assays: His Tagged IL-7 Variants

IL-7 variants fused with an N-terminal His-tag (SEQ ID NO: 20) were expressed and purified using methods known in the art, and the following in vitro experiments were performed.

pSTAT5 Phosphorylation ELISA Assays

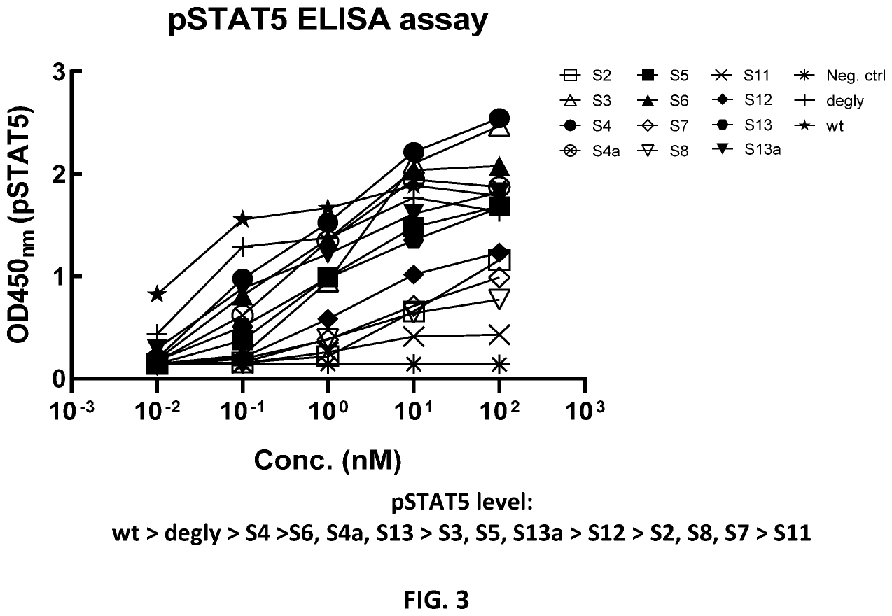

IL-7-induced STAT5 phosphorylation on primary T cells was determined by STAT5 pY694/699 ELISA kit (Abcam, Cat #: ab176656). Specifically, human peripheral blood mononuclear cells (PBMCs) were isolated from healthy human blood, and the total T cells were isolated by MagniSort™ Human T cell Enrichment kit (Invitrogen, Cat #: 8804-6810-74) and cultured in complete RPMI medium containing 10% fetal bovine serum (FBS), and 1× streptomycin/penicillin. The purified IL-7 variants were then serially diluted (10-fold) to final concentrations of 10⁻² nM to 10² nM. 5×10⁵ cells/well T cells were rested for 2 hours and then incubated with the diluted His-tagged IL-7 variant proteins in a low-binding 96-well U-shaped plate at 37° C. for 30 minutes. Lysates of the treated T cells were incubated with the anti-pSTAT5 antibody cocktail (provided in the STAT5 pY694/699 ELISA kit) in a pre-coated plate for 1 hour. After the incubation, 3,3′,5,5′-Tetramethylbenzidine (TMB) was added for color development. The absorbance value at 450 nm (OD450) was measured using a Varioskan™ LUX plate reader (Thermo). As shown in FIG. 3, the tested IL-7 variants induced distinct STAT5 phosphorylation on primary T cells.

### Primary T Cell Proliferation Assays

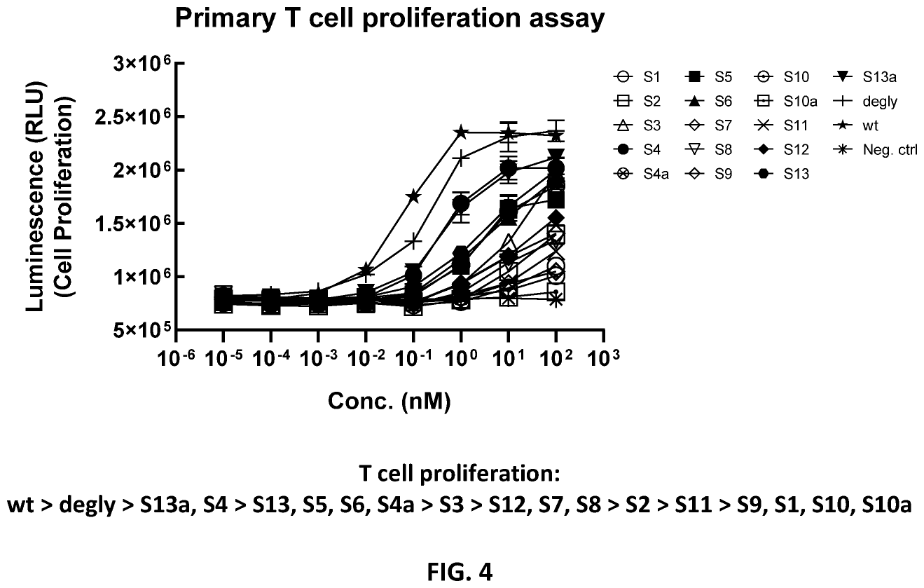

Induction of primary T cell proliferation was performed as follows. PBMCs were isolated from healthy human blood, and the total T cells were isolated by MagniSort™ Human T cell Enrichment kit (Invitrogen, Cat #: 8804-6810-74) and cultured in complete RPMI medium containing 10% FBS, and 1× streptomycin/penicillin. The purified IL-7 variants were then serially diluted (10-fold) to final concentrations of 10⁻⁵ to 10² nM. 5×10⁴ cells/well T cells were incubated with the diluted His-tagged IL-7 variant proteins in a low-binding 96-well U-shaped plate at 37° C. for 7 days. The ability of the IL-7 variant proteins to induce T cell proliferation was evaluated by the CellTiter-Glo® Luminescent Cell Viability Assay (Promega, Cat #: G7573). As shown in FIG. 4, the tested IL-7 variants showed distinct potencies to induce primary T cell proliferation.

### ELISA Binding Assay

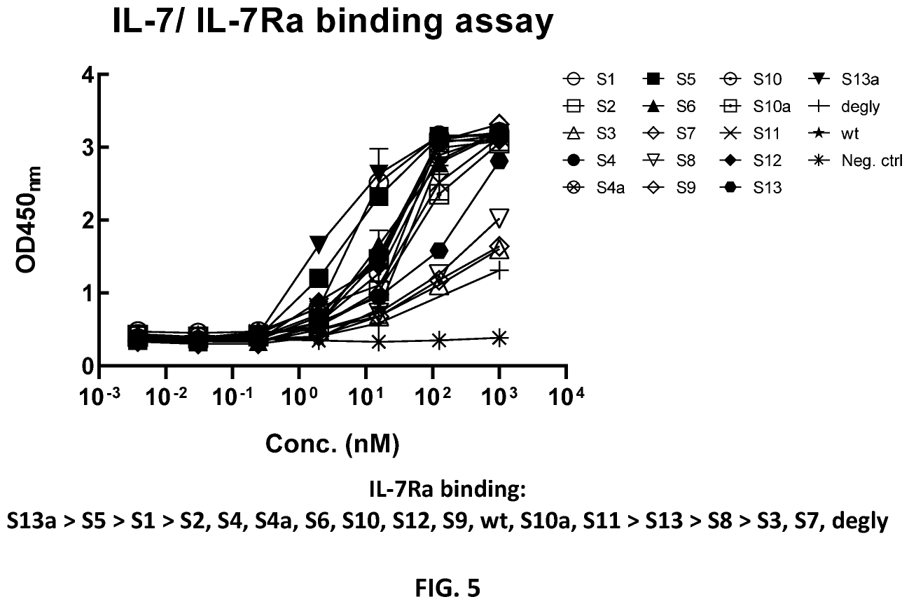

To determine the human CD127 (IL-7Rα) binding ability of the purified IL-7 variant proteins, ELISA plates coated with 1 μg/ml anti-His tag antibody (R&D, Cat #: MAB050-500) were prepared. 30 μl of the serially diluted His-tagged IL-7 variants was added to the ELISA plates and incubated at 25° C. for 1 hour. After the incubation, CD127-Fc (Acrobiosystems, Cat #: ILA-H5258) was added to the ELISA plates and incubated at 25° C. for 1 hour. After the incubation, goat anti-human IgG-HRP (Jackson ImmunoResearch, Cat #: 109-035-098) with a dilution ratio of 1:8000 was added to the plates, and TMB (SURMODICS, Cat #: TMBW-0100-01) was added for color development. The absorbance value at 450 nm (OD450) was measured using a Varioskan™ LUX plate reader (Thermo). As shown in FIG. 5, the tested IL-7 variants showed different binding capacities to CD127.

### Binding Kinetic Analysis

The binding affinity of the His-tagged IL-7 variant proteins to CD127-Fc was also determined by an Octet® Red96 system. The following parameters were used during detection: sensors: anti-Human IgG Fc Capture Biosensors; buffer: 1×PBST buffer supplemented with 1% bovine serum albumin (BSA); ligand: IL-7Rα Fc; loading threshold: 1.0 nm; concentration: 1.23-300 nM (6 concentrations in total by 3-fold dilutions); associate/dissociate time: 60 seconds/60 seconds; association/dissociation time (s1): 120 seconds/180 seconds.

The binding affinity of each protein was determined by calculating its K_{D} value (K_{D}=K_{dis}/Kₒₙ), and the detailed results are shown in the table below.

TABLE 1

| | K_{D} (M) | Kₒₙ(1/Ms) | K_{dis}(1/s) |
|---|---|---|---|
| IL-7_s1 | 1.89E−09 ± 2.45E−10 | 2.20E+05 ± 1.21E+04 | 4.18E−04 ± 7.47E−05 |
| IL-7_s2 | 1.06E−08 | 4.78E+05 | 5.06E−03 |
| IL-7_s4 | 3.36E−08 | 1.23E+06 | 4.01E−02 |
| IL-7_s4a | 1.45E−08 ± 2.87E−10 | 1.87E+06 ± 1.08E+05 | 2.72E−02 ± 2.07E−03 |
| IL-7_s5 | 7.57E−09 ± 1.26E−10 | 2.76E+06 ± 1.67E+05 | 2.09E−02 ± 1.51E−03 |
| IL-7_s6 | 1.71E−08 ± 7.74E−10 | 2.53E+06 ± 1.92E+05 | 4.34E−02 ± 4.94E−03 |
| IL-7_s7 | 5.67E−08 ± 9.41E−09 | 2.33E+06 ± 1.38E+05 | 1.31E−01 ± 1.59E−02 |
| IL-7_s8 | 3.34E−08 | 2.97E+06 | 9.92E−02 |
| IL-7_s9 | 4.93E−08 | 2.60E+06 | 1.29E−01 |
| IL-7_s10 | 3.53E−08 ± 2.44E−09 | 1.99E+06 ± 8.72E+04 | 7.06E−02 ± 7.86E−03 |
| IL-7_s10a | 3.46E−08 ± 2.21E−09 | 2.15E+06 ± 1.54E+05 | 7.46E−02 ± 9.18E−03 |
| IL-7_s11 | 3.93E−08 | 2.02E+06 | 7.94E−02 |
| IL-7_s12 | 2.59E−08 | 2.42E+06 | 6.27E−02 |
| IL-7_s13 | 3.19E−08 ± 1.04E−09 | 2.56E+06 ± 1.35E+05 | 8.17E−02 ± 6.86E−03 |
| IL-7_s13a | 3.60E−09 ± 1.57E−09 | 2.27E+06 ± 9.94E+04 | 8.17E−03 ± 7.12E−04 |
| IL-7 wt-degly | 6.47E−08 | 2.84E+05 | 1.80E−02 |
| IL-7-wt | 1.12E−08 | 5.40E+06 | 6.07E−02 |

The results showed that most of the tested IL-7 variants exhibited a similar binding affinity to CD127 as compared to the wildtype IL-7. Further, the results indicate that the binding capacity of the IL-7 variants was independent of IL-7-mediated functional potency.

### Example 3. In Vitro Assays: His-Tagged IL-7G Variants

The glutamine (Glu, or Q) residues corresponding to positions 95, 116, and/or 141 of SEQ ID NO: 1 in IL-7 variants S4, S4a, S5, S6, S12, S13, and S13a were mutated back to asparagine (Asn, or N). The resultant IL-7 variants were named S4G (SEQ ID NO: 21), S4aG (SEQ ID NO: 22), S5G (SEQ ID NO: 23), S6G (SEQ ID NO: 24), S12G (SEQ ID NO: 25), S13G (SEQ ID NO: 26), and S13aG (SEQ ID NO: 27). Because the glycosylation sites were restored in these IL-7 variants, they were also named as IL-7G variants. Sequences of the IL-7G variants are shown in FIG. 6. The IL-7G variants fused with an N-terminal His-tag (SEQ ID NO: 20) were expressed and purified using methods known in the art, and the following in vitro experiments were performed.

### Primary T Cell Proliferation Assays

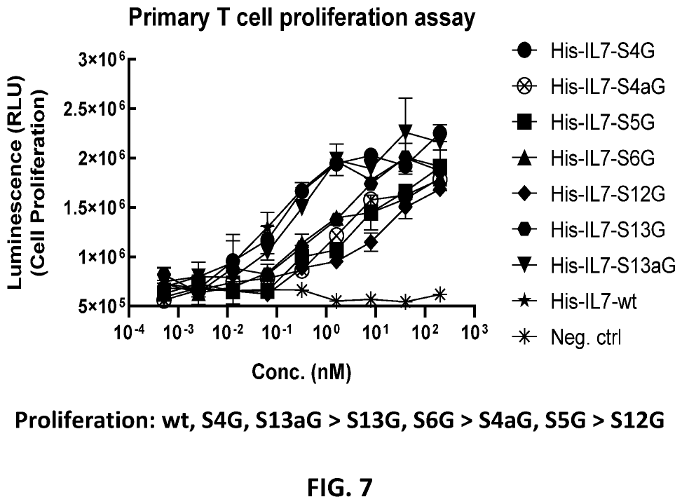

Induction of primary T cell proliferation was performed using the same methods as described herein. As shown in FIG. 7, the tested His-tagged IL-7G variants (His-IL-7G) showed similar potencies to induce primary T cell proliferation as compared to the corresponding IL-7 variants.

### Example 4. In Vitro Assays: Fc-Fused IL-7G Variants

IL-7G variants S4G (SEQ ID NO: 21), S5G (SEQ ID NO: 23), S12G (SEQ ID NO: 25), and S13aG (SEQ ID NO: 27) were fused to the C-terminus of human IgG4 Fc region (SEQ ID NO: 28) via a linker peptide sequence (SEQ ID NO: 29). The human IgG4 Fc region includes a S228P mutation according to EU numbering. The resultant hG4-IL-7G variants (also named as hG4Fc-IL-7G variants) were expressed and purified using methods known in the art, and the following in vitro experiments were performed.

### Primary T Cell Proliferation Assays

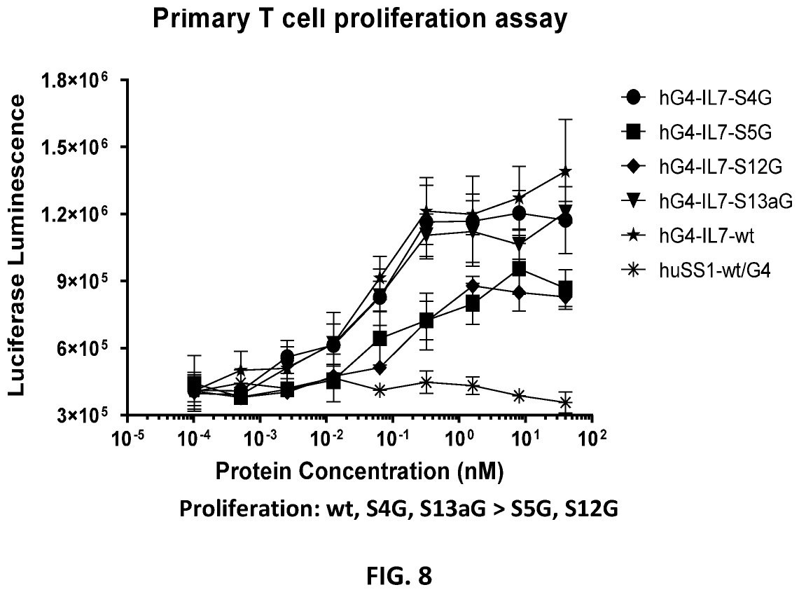

Induction of primary T cell proliferation was performed using the same methods as described herein. As shown in FIG. 8, the tested hG4-IL-7G variants showed similar potencies to induce T cell proliferation as compared to the corresponding IL-7 variants.

### ELISA Binding Assays

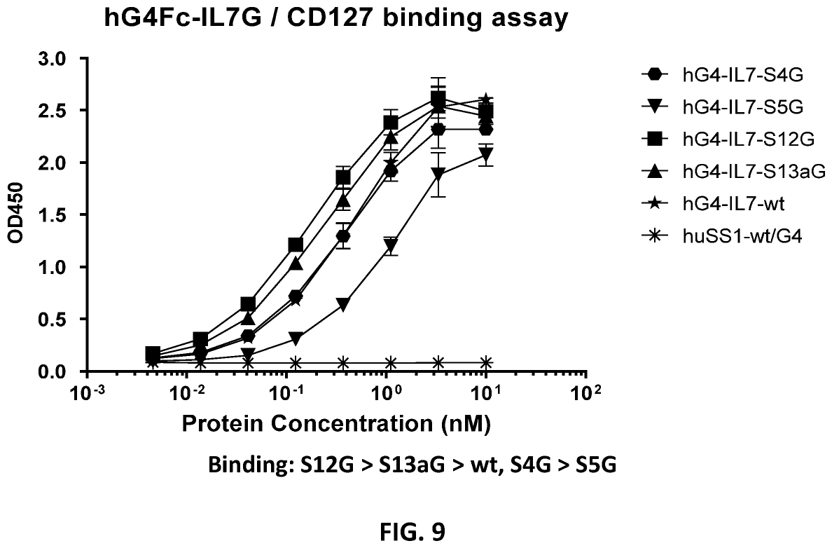

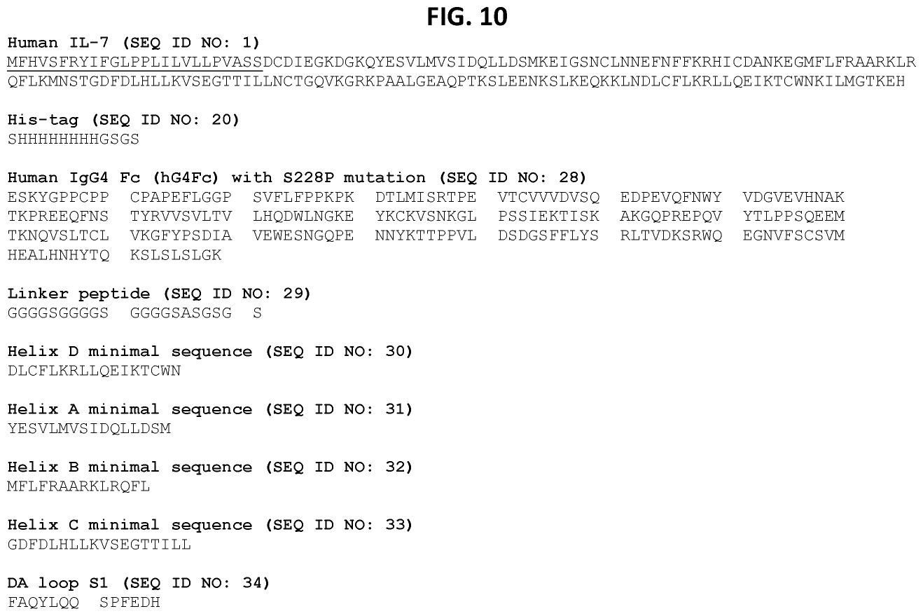

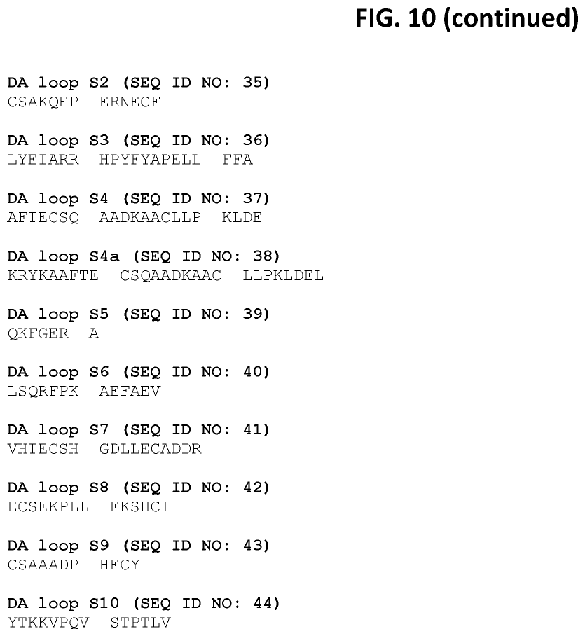

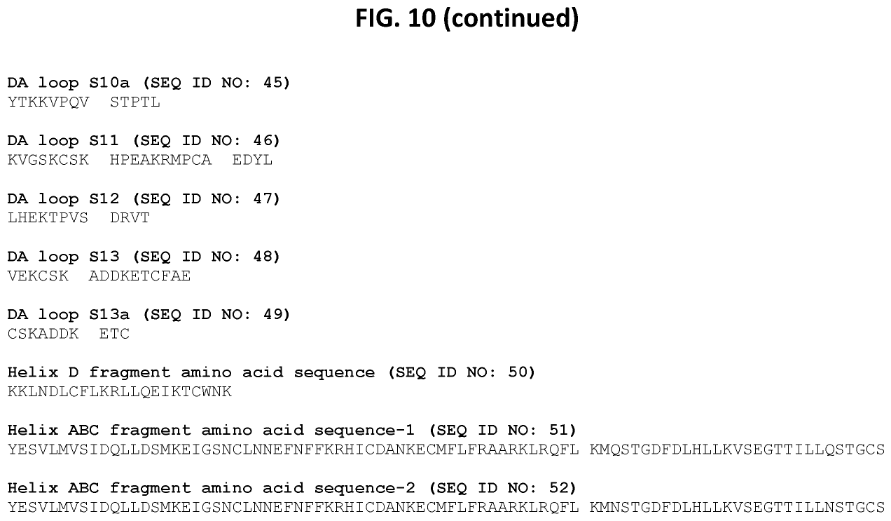

To determine the human CD127 binding ability of the purified hG4-IL-7G variants, ELISA plates coated with 0.5 μg/ml His-CD127 (Sinobiologic, Cat #: 10975-H08H) were prepared. 30 μl of the hG4-IL-7G variants was added to the ELISA plates and incubated at 25° C. for 1 hour. After the incubation, goat anti-human IgG-HRP (Jackson ImmunoResearch, Cat #: 109-035-098) with a dilution ratio of 1:8000 was added to the plates, and TMB (SURMODICS, Cat #: TMBW-0100-01) was added for color development. The absorbance value at 450 nm (OD450) was measured using a Varioskan™ LUX plate reader (Thermo). As shown in FIG. 9, the tested hG4-IL-7G variants showed different binding capacities to CD127. The results indicate that the binding capacity of the hG4-IL-7G variants was independent of IL-7-mediated functional potency.

### Example 5. Evaluation of the Anti-Tumor Efficacy and In Vivo Toxicity of Fc-Fused IL-7 Variants

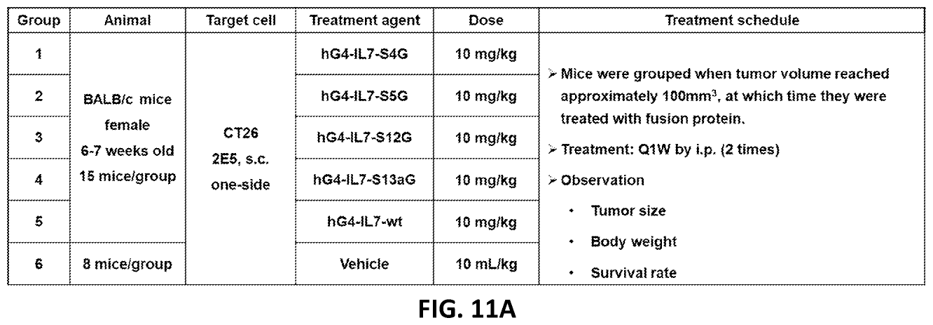

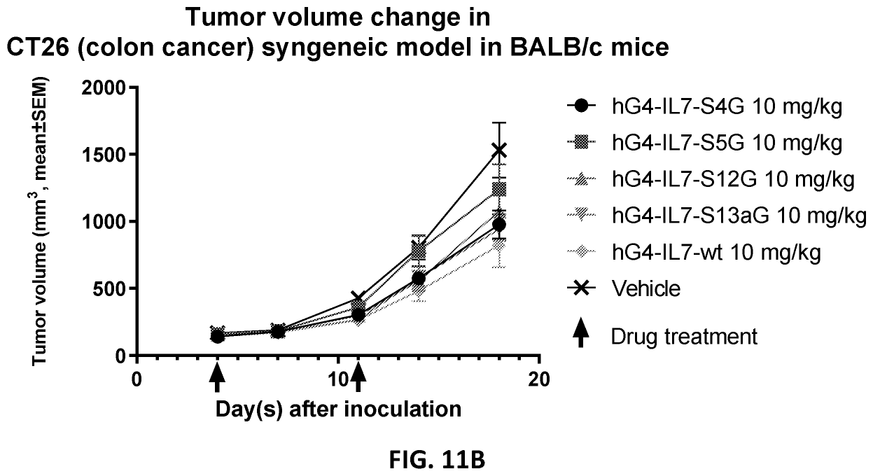

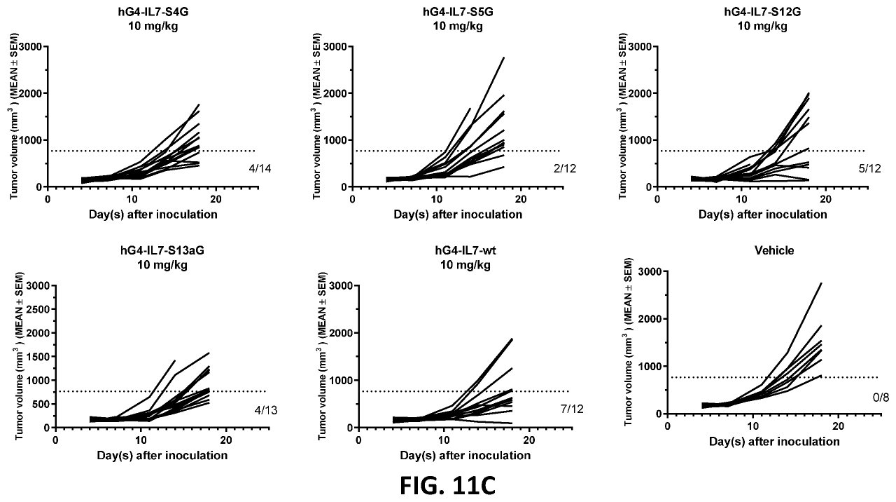

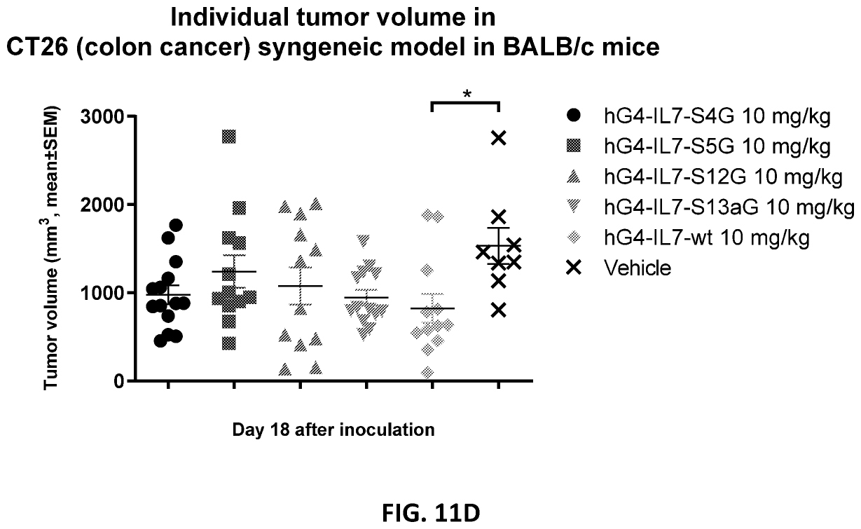

The anti-tumor efficacy of IgG4 Fc-fused IL-7 and its variants were evaluated in a CT26 tumor-bearing BALB/c mouse model. The treatment plan and dosing schedule are shown in FIG. 11A. Specifically, 6-7 weeks old BALB/c mice were selected and subcutaneously (s.c.) inoculated with 2×10⁵ mouse colon cancer cell CT26. When the tumor volume reached about 100-200 mm³ (after 4 days), the mice were randomly placed into 5 treatment groups and 1 control group (15 mice per treatment group and 8 mice pre control group). The treatment group mice were intraperitoneally (i.p.) injected with 10 mg/kg hG4-IL7-S4G, hG4-IL7-S5G, hG4-IL7-S12G, hG4-IL7-S13aG, or hG4-IL7-wt. The control group mice were injected with 10 ml/kg vehicle (isotonic sodium chloride solution). The treatment started on the grouping day (Day 4), and the injections were performed once a week (for a total of 2-3 weeks).

As shown in FIG. 11B, compared to hG4-IL7-S4G, hG4-IL7-S5G, hG4-IL7-S12G, and hG4-IL7-S13aG, hG4-IL7-wt showed significant tumor growth inhibition. Individual tumor growth curves and the tumor volume of each mouse on Day 18 (18 days after inoculation) are shown in FIG. 11C and FIG. 11D, respectively. The ratios next to the tumor growth curves (e.g., “4/14” for hG4-IL7-S4G curves) in FIG. 11C indicate the ratio of mice having a tumor volume that was less than the average half tumor volume of the vehicle control group mice. The results show that the TGI % of hG4-IL7-S4G, hG4-IL7-S13aG, and hG4-IL7-wt is 39%, 43%, and 50%, respectively, in the CT26 syngeneic mouse model in Table 2. The corresponding P value for hG4-IL7-wt was determined to be less than 0.05. The results indicate that hG4-IL7-wt induced remarkable tumor growth inhibition in the CT26 syngeneic mouse model. The hG4-IL7-S4G and hG4-IL7-S13aG induced slight tumor growth inhibition.

TABLE 2

| | | | P value |
|---|---|---|---|
| | | | One-way ANOVA followed |
| D18 after | Mean ± SEM | | by Bonferroni's multiple |
| inoculation | (mm³) | TGI % | comparisons test |
| hG4-IL7-S4G | 977 ± 106 | 39% | 0.130 |
| 10 mg/kg | | | |
| hG4-IL7-S5G | 1239 ± 187 | 21% | 0.999 |
| 10 mg/kg | | | |
| hG4-IL7-S12G | 1078 ± 211 | 33% | 0.374 |
| 10 mg/kg | | | |
| hG4-IL7-S13aG | 943 ± 88 | 43% | 0.100 |
| 10 mg/kg | | | |
| hG4-IL7-WT | 822 ± 162 | 50% | 0.031 |
| 10 mg/kg | | | |
| Vehicle | 1530 ± 206 | — | — |

Survival rate after treatment results in Table 3 below suggest that treatment of hG4-IL7-S4G and hG4-IL7-S13aG induced less toxicity than hG4-IL7-wt treatment on CT26 syngeneic mice.

TABLE 3

| | S4G | S5G | S12G | S13aG | wt | Vehicle |
|---|---|---|---|---|---|---|
| Died/Total mouse no. | 1/15 | 2/15 | 3/15 | 1/15 | 3/15 | 0/8 |
| after treatment | | | | | | |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An engineered IL-7 polypeptide comprising: a helix D, a helix A, a helix B, and a helix C, wherein the helix D and helix A are linked via a DA loop sequence.

2. The engineered IL-7 polypeptide of claim 1, wherein the engineered IL-7 polypeptide maintains the orientation of helix A, helix B, helix C, and helix D of a wildtype IL-7 polypeptide.

3. The engineered IL-7 polypeptide of any one of claims 1-2, wherein the engineered IL-7 polypeptide can bind to CD127 (e.g., human CD127) and/or CD132 (e.g., human CD132).

4. The engineered IL-7 polypeptide of any one of claims 1-3, wherein the engineered IL-7 polypeptide can induce STAT5 phosphorylation on primary T cells and/or induce primary T cell proliferation.

5. The engineered IL-7 polypeptide of any one of claims 1-4, wherein the engineered IL-7 polypeptide comprises from N-terminus to C-terminus:
(a) a helix D fragment amino acid sequence; and
(b) a helix ABC fragment amino acid sequence,
wherein the helix D fragment and the helix ABC fragment are linked by the DA loop sequence.

6. The engineered IL-7 polypeptide of claim 5, wherein the helix D fragment amino acid sequence is at least 80%, 85%, 90%, or 95% identical to SEQ ID NO: 50 and/or the helix ABC fragment amino acid sequence is at least 80%, 85%, 90%, or 95% identical to SEQ ID NO: 51 or SEQ ID NO: 52.

7. An engineered IL-7 polypeptide comprising from N-terminus to C-terminus:
(a) a helix D sequence that is at least 80%, 85%, 90%, or 95% identical to SEQ ID NO: 30;
(b) a helix A sequence that is at least 80%, 85%, 90%, or 95% identical to SEQ ID NO: 31;
(c) a helix B sequence that is at least 80%, 85%, 90%, or 95% identical to SEQ ID NO: 32; and
(d) a helix C sequence that is at least 80%, 85%, 90%, or 95% identical to SEQ ID NO: 33.

8. The engineered IL-7 polypeptide of claim 7, comprising a DA loop sequence between the helix D sequence and the helix A sequence, wherein the DA loop sequence is at least 80%, 85%, 90%, or 95% identical to any one of SEQ ID NOs: 34-49.

9. The engineered IL-7 polypeptide of claim 7 or 8, wherein the engineered IL-7 polypeptide comprises an amino acid sequence that is at least 80%, 85%, 90%, or 95% identical to SEQ ID NO: 50.

10. The engineered IL-7 polypeptide of any one of claims 7-9, wherein the engineered IL-7 polypeptide comprises an amino acid sequence that is at least 80%, 85%, 90%, or 95% identical to SEQ ID NO: 51.

11. The engineered IL-7 polypeptide of any one of claims 7-10, wherein the engineered IL-7 polypeptide comprises an amino acid sequence that is at least 80%, 85%, 90%, or 95% identical to any one of SEQ ID NOs: 3-18.

12. The engineered IL-7 polypeptide of any one of claims 7-9, wherein the engineered IL-7 polypeptide comprises an amino acid sequence that is at least 80%, 85%, 90%, or 95% identical to SEQ ID NO: 52.

13. The engineered IL-7 polypeptide of any one of claims 7-9 and 12, wherein the engineered IL-7 polypeptide comprises an amino acid sequence that is at least 80%, 85%, 90%, or 95% identical to any one of SEQ ID NOs: 21-27.

14. An engineered IL-7 polypeptide, wherein the engineered IL-7 polypeptide comprises mutations at one or more glycosylation sites.

15. The engineered IL-7 polypeptide of claim 14, comprising one or more of the following:
(a) the amino acid that corresponds to N95 of SEQ ID NO: 1 is Q, E, or A;
(b) the amino acid that corresponds to N116 of SEQ ID NO: 1 is Q, E, or A; and/or
(c) the amino acid that corresponds to N141 of SEQ ID NO: 1 is Q, E, or A.

16. The engineered IL-7 polypeptide of claim 14 or 15, comprising one or more of the following:
(a) the amino acid that corresponds to N95 of SEQ ID NO: 1 is Q;
(b) the amino acid that corresponds to N116 of SEQ ID NO: 1 is Q; and/or
(c) the amino acid that corresponds to N141 of SEQ ID NO: 1 is Q.

17. The engineered IL-7 polypeptide of any one of claims 14-16, wherein the engineered IL-7 polypeptide comprises an amino acid sequence that is at least 80%, 85%, 90%, or 95% identical to SEQ ID NO: 19 or SEQ ID NO: 51.

18. A protein construct comprising the engineered IL7 polypeptide of any one of claims 1-17.

19. The protein construct of claim 18, comprising two or more engineered IL-7 polypeptides.

20. The protein construct of claim 19, wherein at least two engineered IL-7 polypeptides are identical.

21. The protein construct of claim 19, wherein at least two engineered IL-7 polypeptides are different.

22. The protein construct of any one of claims 18-21, further comprising an Fc region.

23. The protein construct of claim 22, wherein the Fc region is an IgG4 Fc region (e.g., a human IgG4 Fc region with a S228P mutation according to EU numbering).

24. The protein construct of claim 22 or 23, wherein the Fc region comprises an amino acid sequence that is at least 80%, 85%, 90%, or 95% identical to SEQ ID NO: 28.

25. The protein construct of claim 22, wherein the Fc region is an IgG1 Fc region (e.g., with LALA mutations or LALA-PG mutations).

26. The protein construct of any one of claims 22-25, wherein the engineered IL-7 polypeptide is connected to the Fc region via a linker peptide.

27. The protein construct of claim 26, wherein the linker peptide comprises an amino acid sequence that is at least 80%, 85%, 90%, or 95% identical to SEQ ID NO: 29.

28. A protein construct comprising
a first fusion polypeptide comprising the engineered IL-7 polypeptide of any one of claims 1-17, a first CH2 domain, and a first CH3 domain; and
a second fusion polypeptide comprising a second CH2 domain, and a second CH3 domain;
wherein the first fusion polypeptide and the second fusion polypeptide associate with each other, forming a dimer.

29. The protein construct of claim 28, wherein the second fusion polypeptide further comprises a second engineered IL-7 polypeptide.

30. A pharmaceutical composition comprising the engineered IL-7 polypeptide of any one of claims 1-17 or the protein construct of any one of claims 18-29; and
a pharmaceutically acceptable carrier.

31. A nucleic acid encoding the engineered IL-7 polypeptide of any one of claims 1-17 or the protein construct of any one of claims 18-29.

32. A vector comprising the nucleic acid of claim 31.

33. A cell comprising the nucleic acid of claim 31.

34. The cell of claim 33, wherein the cell is a CHO cell.

35. A method of producing an engineered IL-7 polypeptide or a protein construct comprising the engineered IL-7 polypeptide, the method comprising
(a) culturing the cell of claim 33 or 34 under conditions sufficient for the cell to produce the engineered IL-7 polypeptide or the protein construct; and
(b) collecting the engineered IL-7 polypeptide or the protein construct produced by the cell.

36. A method of treating a subject having cancer, the method comprising administering a therapeutically effective amount of a composition comprising the engineered IL-7 polypeptide of any one of claims 1-17 or the protein construct of any one of claims 18-29, to the subject.

37. The method of claim 36, wherein the subject has a solid tumor or a hematologic cancer.

38. The method of claim 36, wherein the cancer is lung cancer, melanoma, colorectal cancer, glioma, pancreatic cancer, lymphoma, leukemia, prostate cancer, renal cell carcinoma (RCC), hepatocellular carcinoma, cholangiocarcinoma, gallbladder cancer, gastric cancer, endometrial carcinoma, ovarian cancer, bladder cancer, or glioblastoma.

39. A method of decreasing the rate of tumor growth, the method comprising contacting a tumor cell with an effective amount of a composition comprising the engineered IL-7 polypeptide of any one of claims 1-17 or the protein construct of any one of claims 18-29.

40. A method of killing a tumor cell, the method comprising contacting a tumor cell with an effective amount of a composition comprising the engineered IL-7 polypeptide of any one of claims 1-17 or the protein construct of any one of claims 18-29.


SEQUENCE LISTING (52 sequences)

SEQ ID NO: 1 - protein, 177 aa, Homo sapiens
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 2 - protein, 152 aa, Homo sapiens
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 3 - protein, 123 aa, synthetic construct
KKLNDLCFLKRLLQEIKTCWNKIFAQYLQQSPFEDHKQYESVLMVSIDQLLDSMKEIGSNCLNNEFNFFKRHICDANKECMFLFRAARKLRQFLKMQSTGDFDLHLLKVSEGTTILLQSTGCS

SEQ ID NO: 4 - protein, 123 aa, synthetic construct
KKLNDLCFLKRLLQEIKTCWNKICSAKQEPERNECFKQYESVLMVSIDQLLDSMKEIGSNCLNNEFNFFKRHICDANKECMFLFRAARKLRQFLKMQSTGDFDLHLLKVSEGTTILLQSTGCS

SEQ ID NO: 5 - protein, 130 aa, synthetic construct
KKLNDLCFLKRLLQEIKTCWNKILYEIARRHPYFYAPELLFFAKQYESVLMVSIDQLLDSMKEIGSNCLNNEFNFFKRHICDANKECMFLFRAARKLRQFLKMQSTGDFDLHLLKVSEGTTILLQSTGCS

SEQ ID NO: 6 - protein, 129 aa, synthetic construct
KKLNDLCFLKRLLQEIKTCWNKIAFTECSQAADKAACLLPKLDEYESVLMVSIDQLLDSMKEIGSNCLNNEFNFFKRHICDANKECMFLFRAARKLRQFLKMQSTGDFDLHLLKVSEGTTILLQSTGCS

SEQ ID NO: 7 - protein, 135 aa, synthetic construct
KKLNDLCFLKRLLQEIKTCWNKRYKAAFTECSQAADKAACLLPKLDELKQYESVLMVSIDQLLDSMKEIGSNCLNNEFNFFKRHICDANKECMFLFRAARKLRQFLKMQSTGDFDLHLLKVSEGTTILLQSTGCS

SEQ ID NO: 8 - protein, 119 aa, synthetic construct
KKLNDLCFLKRLLQEIKTCWNKILQKFGERAAKQYESVLMVSIDQLLDSMKEIGSNCLNNEFNFFKRHICDANKECMFLFRAARKLRQFLKMQSTGDFDLHLLKVSEGTTILLQSTGCS

SEQ ID NO: 9 - protein, 123 aa, synthetic construct
KKLNDLCFLKRLLQEIKTCWNKILSQRFPKAEFAEVKQYESVLMVSIDQLLDSMKEIGSNCLNNEFNFFKRHICDANKECMFLFRAARKLRQFLKMQSTGDFDLHLLKVSEGTTILLQSTGCS

SEQ ID NO: 10 - protein, 127 aa, synthetic construct
KKLNDLCFLKRLLQEIKTCWNKIVHTECSHGDLLECADDRKQYESVLMVSIDQLLDSMKEIGSNCLNNEFNFFKRHICDANKECMFLFRAARKLRQFLKMQSTGDFDLHLLKVSEGTTILLQSTGCS

SEQ ID NO: 11 - protein, 123 aa, synthetic construct
KKLNDLCFLKRLLQEIKTCWNKECSEKPLLEKSHCIKQYESVLMVSIDQLLDSMKEIGSNCLNNEFNFFKRHICDANKECMFLFRAARKLRQFLKMQSTGDFDLHLLKVSEGTTILLQSTGCS

SEQ ID NO: 12 - protein, 121 aa, synthetic construct
KKLNDLCFLKRLLQEIKTCWNKICSAAADPHECYKQYESVLMVSIDQLLDSMKEIGSNCLNNEFNFFKRHICDANKECMFLFRAARKLRQFLKMQSTGDFDLHLLKVSEGTTILLQSTGCS

SEQ ID NO: 13 - protein, 122 aa, synthetic construct
KKLNDLCFLKRLLQEIKTCWNKYTKKVPQVSTPTLVQYESVLMVSIDQLLDSMKEIGSNCLNNEFNFFKRHICDANKECMFLFRAARKLRQFLKMQSTGDFDLHLLKVSEGTTILLQSTGCS

SEQ ID NO: 14 - protein, 122 aa, synthetic construct
KKLNDLCFLKRLLQEIKTCWNKYTKKVPQVSTPTLKQYESVLMVSIDQLLDSMKEIGSNCLNNEFNFFKRHICDANKECMFLFRAARKLRQFLKMQSTGDFDLHLLKVSEGTTILLQSTGCS

SEQ ID NO: 15 - protein, 131 aa, synthetic construct
KKLNDLCFLKRLLQEIKTCWNKKVGSKCSKHPEAKRMPCAEDYLKQYESVLMVSIDQLLDSMKEIGSNCLNNEFNFFKRHICDANKECMFLFRAARKLRQFLKMQSTGDFDLHLLKVSEGTTILLQSTGCS

SEQ ID NO: 16 - protein, 121 aa, synthetic construct
KKLNDLCFLKRLLQEIKTCWNKLHEKTPVSDRVTKQYESVLMVSIDQLLDSMKEIGSNCLNNEFNFFKRHICDANKECMFLFRAARKLRQFLKMQSTGDFDLHLLKVSEGTTILLQSTGCS

SEQ ID NO: 17 - protein, 129 aa, synthetic construct
KKLNDLCFLKRLLQEIKTCWNKILVEKCSKADDKETCFAEDGKQYESVLMVSIDQLLDSMKEIGSNCLNNEFNFFKRHICDANKECMFLFRAARKLRQFLKMQSTGDFDLHLLKVSEGTTILLQSTGCS

SEQ ID NO: 18 - protein, 120 aa, synthetic construct
KKLNDLCFLKRLLQEIKTCWNKICSKADDKETCKQYESVLMVSIDQLLDSMKEIGSNCLNNEFNFFKRHICDANKECMFLFRAARKLRQFLKMQSTGDFDLHLLKVSEGTTILLQSTGCS

SEQ ID NO: 19 - protein, 152 aa, synthetic construct
DCDIEGKDGKQYESVLMVSIDQLLDSMKEIGSNCLNNEFNFFKRHICDANKEGMFLFRAARKLRQFLKMQSTGDFDLHLLKVSEGTTILLQCTGQVKGRKPAALGEAQPTKSLEEQKSLKEQKKLNDLCFLKRLLQEIKTCWNKILMGTKEH

SEQ ID NO: 20 - protein, 13 aa, synthetic construct
SHHHHHHHHGSGS

SEQ ID NO: 21 - protein, 129 aa, synthetic construct
KKLNDLCFLKRLLQEIKTCWNKIAFTECSQAADKAACLLPKLDEYESVLMVSIDQLLDSMKEIGSNCLNNEFNFFKRHICDANKECMFLFRAARKLRQFLKMNSTGDFDLHLLKVSEGTTILLNSTGCS

SEQ ID NO: 22 - protein, 135 aa, synthetic construct
KKLNDLCFLKRLLQEIKTCWNKRYKAAFTECSQAADKAACLLPKLDELKQYESVLMVSIDQLLDSMKEIGSNCLNNEFNFFKRHICDANKECMFLFRAARKLRQFLKMNSTGDFDLHLLKVSEGTTILLNSTGCS

SEQ ID NO: 23 - protein, 119 aa, synthetic construct
KKLNDLCFLKRLLQEIKTCWNKILQKFGERAAKQYESVLMVSIDQLLDSMKEIGSNCLNNEFNFFKRHICDANKECMFLFRAARKLRQFLKMNSTGDFDLHLLKVSEGTTILLNSTGCS

SEQ ID NO: 24 - protein, 123 aa, synthetic construct
KKLNDLCFLKRLLQEIKTCWNKILSQRFPKAEFAEVKQYESVLMVSIDQLLDSMKEIGSNCLNNEFNFFKRHICDANKECMFLFRAARKLRQFLKMNSTGDFDLHLLKVSEGTTILLNSTGCS

SEQ ID NO: 25 - protein, 121 aa, synthetic construct
KKLNDLCFLKRLLQEIKTCWNKLHEKTPVSDRVTKQYESVLMVSIDQLLDSMKEIGSNCLNNEFNFFKRHICDANKECMFLFRAARKLRQFLKMNSTGDFDLHLLKVSEGTTILLNSTGCS

SEQ ID NO: 26 - protein, 129 aa, synthetic construct
KKLNDLCFLKRLLQEIKTCWNKILVEKCSKADDKETCFAEDGKQYESVLMVSIDQLLDSMKEIGSNCLNNEFNFFKRHICDANKECMFLFRAARKLRQFLKMNSTGDFDLHLLKVSEGTTILLNSTGCS

SEQ ID NO: 27 - protein, 120 aa, synthetic construct
KKLNDLCFLKRLLQEIKTCWNKICSKADDKETCKQYESVLMVSIDQLLDSMKEIGSNCLNNEFNFFKRHICDANKECMFLFRAARKLRQFLKMNSTGDFDLHLLKVSEGTTILLNSTGCS

SEQ ID NO: 28 - protein, 229 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 29 - protein, 21 aa, synthetic construct
GGGGSGGGGSGGGGSASGSGS

SEQ ID NO: 30 - protein, 17 aa, synthetic construct
DLCFLKRLLQEIKTCWN

SEQ ID NO: 31 - protein, 16 aa, synthetic construct
YESVLMVSIDQLLDSM

SEQ ID NO: 32 - protein, 14 aa, synthetic construct
MFLFRAARKLRQFL

SEQ ID NO: 33 - protein, 18 aa, synthetic construct
GDFDLHLLKVSEGTTILL

SEQ ID NO: 34 - protein, 13 aa, synthetic construct
FAQYLQQSPFEDH

SEQ ID NO: 35 - protein, 13 aa, synthetic construct
CSAKQEPERNECF

SEQ ID NO: 36 - protein, 20 aa, synthetic construct
LYEIARRHPYFYAPELLFFA

SEQ ID NO: 37 - protein, 21 aa, synthetic construct
AFTECSQAADKAACLLPKLDE

SEQ ID NO: 38 - protein, 27 aa, synthetic construct
KRYKAAFTECSQAADKAACLLPKLDEL

SEQ ID NO: 39 - protein, 7 aa, synthetic construct
QKFGERA

SEQ ID NO: 40 - protein, 13 aa, synthetic construct
LSQRFPKAEFAEV

SEQ ID NO: 41 - protein, 17 aa, synthetic construct
VHTECSHGDLLECADDR

SEQ ID NO: 42 - protein, 14 aa, synthetic construct
ECSEKPLLEKSHCI

SEQ ID NO: 43 - protein, 11 aa, synthetic construct
CSAAADPHECY

SEQ ID NO: 44 - protein, 14 aa, synthetic construct
YTKKVPQVSTPTLV

SEQ ID NO: 45 - protein, 13 aa, synthetic construct
YTKKVPQVSTPTL

SEQ ID NO: 46 - protein, 22 aa, synthetic construct
KVGSKCSKHPEAKRMPCAEDYL

SEQ ID NO: 47 - protein, 12 aa, synthetic construct
LHEKTPVSDRVT

SEQ ID NO: 48 - protein, 16 aa, synthetic construct
VEKCSKADDKETCFAE

SEQ ID NO: 49 - protein, 10 aa, synthetic construct
CSKADDKETC

SEQ ID NO: 50 - protein, 22 aa, synthetic construct
KKLNDLCFLKRLLQEIKTCWNK

SEQ ID NO: 51 - protein, 85 aa, synthetic construct
YESVLMVSIDQLLDSMKEIGSNCLNNEFNFFKRHICDANKECMFLFRAARKLRQFLKMQSTGDFDLHLLKVSEGTTILLQSTGCS

SEQ ID NO: 52 - protein, 85 aa, synthetic construct
YESVLMVSIDQLLDSMKEIGSNCLNNEFNFFKRHICDANKECMFLFRAARKLRQFLKMNSTGDFDLHLLKVSEGTTILLNSTGCS